(19) United States
(12) Patent Application Publication
(10) Pub. No.: US 2010/0009015 A1
(43) Pub. Date: Jan. 14, 2010

(54) USE OF CHROMIUM HISTIDINATE FOR TREATMENT OF CARDIOMETABOLIC DISORDERS

(76) Inventors: Vijaya Juturu, Dobbs Ferry, NY (US); James Komorowski, Trumbull, CT (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/512,430

(22) Filed: Jul. 30, 2009

Related U.S. Application Data
(63) Continuation of application No. PCT/US2008/052352, filed on Jan. 29, 2008.
(60) Provisional application No. 60/887,561, filed on Jan. 31, 2007.

(51) Int. Cl. A61K 31/555; A61K 33/24; A61P 3/00

(52) U.S. Cl. 424/655; 514/184

(57) ABSTRACT

Provided herein are methods for treating, preventing, and improving conditions associated with cardiometabolic syndrome, by identifying a subject in need of treatment, prevention, or improvement of a condition associated with cardiometabolic syndrome, and providing a therapeutically effective amount of a composition comprising chromium and histidine, chromium histidinate complexes, or combinations thereof, to the individual.

### CROSS-REFERENCE TO RELATED APPLICATIONS

The present application is a continuation of International Patent Application No. PCT/US2008/052352, filed Jan. 29, 2008, which designated the United States and was published in English, which claims priority under 35 U.S.C. §119(a)-(d) to U.S. Provisional Application No. 60/887,561 to Juturu et al., entitled “USE OF CHROMIUM HISTIDINATE FOR TREATMENT OF CARDIOMETABILIC DISORDERS” filed on Jan. 31, 2007, each of which is incorporated by reference in its entirety.

### BACKGROUND OF THE INVENTION

1. Field of the Invention

Embodiments disclosed herein relate to the use of compositions comprising, consisting essentially of, or consisting of chromium and histidine, chromium histidinate complex, chromium trihistidinate, or chromium polyhistidinate complex, or combinations thereof, including pharmaceutically acceptable salts, hydrates, solvates, or mixtures thereof for the treatment of cardiometabolic syndrome and related conditions, diseases, and disorders.

2. Description of the Related Art

### Cardiometabolic Syndrome

Cardiometabolic syndrome (CMS) describes a constellation of maladaptive cardiovascular, renal, metabolic, prothrombotic, and inflammatory abnormalities. CMS is recognized as a disease entity by the American Society of Endocrinology, National Cholesterol Education Program, and World Health Organization, and is characterized by various salient features such as obesity, hypertension, dyslipidemia, impaired glucose tolerance, increase in inflammatory markers such as C-reactive protein (CRP), cytokines, tumor necrosis factor alpha (TNFα), inteleukins 6 and 10 (IL-6 and IL-10), changes in cell adhesion molecules, prothrombotic and fibrinolytic changes, increase in oxidative stress and endothelial dysfunction. Juturu, 2006 DPG Medical Nutrition Therapy. Several of the conditions associated with CMS, e.g. obesity, hyperlipidemia, and diabetes, play a causal role in atherosclerotic cardiovascular diseases, which currently account for a considerable proportion of mortality and morbidity in developed, developing and underdeveloped societies.

Insulin resistance is the underlying cause for various risk factors for heart attacks, which also lead to cardiometabolic syndrome (CMS). As such, it is not surprising that patients presenting with multiple cardiometabolic risk factors have triple the risk of experiencing a myocardial infarction and/or stroke and double the risk of mortality. In addition, the risk for developing type 2 diabetes, if not already present, is fivefold above the risk in patients without CMS.

In addition to the risks associated with heart attack and stroke, hyperinsulinemia and hypertension, two conditions associated with CMS, can also contribute significantly to progressive renal disease. Other mechanisms that potentially lead to progressive renal disease and CMS can include endothelial dysfunction, left ventricular hypertrophy (LVH), cardiac hyperreactivity, dyslipidemia, hyperglycemia, enhanced renin-angiotensin-aldosterone system (RAAS) activity, altered renal structure and function with impaired pressure natriuresis leading to sodium retention, volume expansion, progressive renal disease, and eventually end-stage renal disease (ESRD).

It has been suggested that the impact of CMS is associated with several neglected modifiable and non modifiable risk factors, such as abdominal obesity, especially visceral obesity. A common pathophysiologic process, such as endothelial dysfunction, chronic low-grade inflammation, or increased transvascular leakage of macromolecules, can underlie the association between microalbuminuria and cardiovascular disease. Microalbuminuria has been implicated as an independent risk factor for CVD and premature cardiovascular mortality for patients with type 1 and type 2 diabetes mellitus, as well as for patients with essential hypertension. The combination of diabetes and CHD risk factors could be explained by metabolic abnormalities that are not currently assessed in daily clinical practice. It is therefore suggested that in order to optimally manage these risk factors, attention should be given not only to reduce risk factors, but also to the improvement of features of the CMS Juturu, 2006 DPGMNT.

Insulin resistance is a condition that is characterized by decreased insulin function and hyperinsulinemia. Individuals who have insulin resistance also have an increased risk of developing diabetes mellitus, dyslipidemia, hypertension, atherosclerosis, endothelial dysfunction, microalbuminuria, obesity, depression, Syndrome X, and polycystic ovary syndrome, among other conditions. In addition, all of the aforementioned conditions carry the risk of developing associated diseases. For example, diabetes increases the risk of developing associated diseases such as diabetic nephropathy, neuropathy, and retinopathy.

Insulin resistance may result from taking certain drug therapies such as statins, non-steroidal anti-inflammatory drugs (NSAIDS), steroids, oral contraceptives, hormone replacement therapy (HRT), beta blockers, potassium channel openers, diuretics, immunosuppressive drugs, etc. For example, A. Jula et al. report that fasting serum insulin levels increased 13% and insulin resistance increased by 14% in 120 nondiabetic hypercholesterolemic male patients taking statin drugs to reduce their cholesterol levels. A. Jula et al., 2002, JAMA 287:598-605, 604 . Furthermore, it has also been reported that beta blockers and diuretics worsen insulin resistance and that patients taking beta blockers had a 28% higher incidence of diabetes than untreated patients with hypertension. S. Julius et al., 2001, Am. J. Hypertens. 14:310S-316S, 313S.

Insulin resistance has also been described as a side effect of a variety of oral contraceptives. In a study of the metabolic effects of implantable steroid contraceptives, altered glucose tolerance characterized by decreased insulin sensitivity following glucose administration was seen in individuals with implantable contraceptives, such as NORPLANT®, JADELLE®, and IMPLANON® was observed. Dorfgliner, L. J., 2002, Contraception 65:47-62, Peterson, K. R., 2002, Danish Medical Billetin 49:43-60. Similarly, oral contraceptives and hormone replacement therapy (“HRT”) have been linked to the onset of microalbuminuria. Monster, T. B. M et al., 2001, Arch Intern Med. 161:2000-2005.

Physicians generally prescribe a hypoglycemic drug such as metformin, which the patient must continue to take for the rest of the patient's life, for individuals presenting with insulin resistance.

### Atherosclerosis

Atherosclerosis is a slowly progressive disease characterized by the accumulation of cholesterol within the arterial wall. Without wishing to be bound by any particular theory and solely for the purposes of expanding knowledge in the field, it is thought that lipids deposited in atherosclerotic lesions are derived primarily from plasma apolipoprotein B (apo B)-containing lipoproteins, which include chylomicrons, very low density lipoproteins (VLDL), intermediate-density lipoproteins (IDL), and LDL. Apo B-containing lipoproteins, and in particular LDL, are associated with adverse health outcomes. By contrast, HDL serum levels, correlate inversely with coronary heart disease. Indeed, high serum levels of HDL are regarded as a negative risk factor for CHD, and studies suggest that high levels of plasma HDL are not only protective against coronary artery disease, but may actually induce regression of atherosclerotic plaque. See, e.g. Badimon et al., 1992 Circulation 86:(Suppl. III) 86 94; Dansky and Fisher, 1999, Circulation 100:1762 3. Data also suggest that non-HDL cholesterol (non HDL-C) might be a better predictive risk factor of CVD than LDL-C. The Adult Treatment Panel (ATP-III) recommended using non-HDL-C in assessing CVD risk in patients with Type II Diabetes Mellitus.

### Cholesterol

As discussed above, elevated serum cholesterol is linked to coronary heart disease. Circulating cholesterol is carried by plasma lipoproteins, which are particles of complex lipid and protein composition that transport lipids in the blood. Low density lipoprotein (LDL) and high density lipoprotein (HDL) are the major cholesterol-carrier proteins. LDL is believed to be responsible for the delivery of cholesterol from the liver, where it is synthesized or obtained from dietary sources, to extrahepatic tissues in the body. “Reverse cholesterol transport” refers to the transport of cholesterol from extrahepatic tissues to the liver, where it is catabolized and eliminated. It is believed that plasma HDL particles play a major role in the reverse transport process, acting as scavengers of tissue cholesterol. HDL is also responsible for the removal of non-cholesterol lipid, oxidized cholesterol and other oxidized products from the bloodstream. The atherogenic index of plasma (AIP), defined as logarithm [log] of the ratio of plasma concentration of triglycerides (TG) to HDL-cholesterol (TG/HDL-C), has recently been proposed as a predictive marker for plasma atherogenicity and is positively correlated with cardiovascular disease (CVD). Lipoprotein subclass abnormalities that accompany insulin resistance are characterized by large, triglyceride-enriched very low-density lipoprotein (VLDL) particles; small, cholesterol-depleted LDL particles; and small HDL particles. In addition, more severe states of insulin resistance have been associated with progressively higher numbers of VLDL particles, intermediate-density lipoprotein particles and, most importantly, LDL particles. The strong correlation of atherogenic index in plasma with lipoprotein particle size may explain its association with cardiovascular disease (CVD) risk. Atherogenic dyslipidemia results in increased atherosclerotic plaque formation because of an imbalance between an increased number of small, dense LDL particles, which carry cholesterol to the vascular endothelium, and a decreased number of HDL particles, which remove cholesterol from atherosclerotic vessels. Insulin resistance is the initial physiological defect in the pathogenesis of diabetes, such as Type II diabetes mellitus (“T2DM”); the associated atherogenic lipoprotein phenotype considerably enhances the risk of CVD. The combination of all these factors may lead to cardiometabolic syndrome which is different from metabolic syndrome. Hyperinsulinemia is often clustered with other cardiovascular risk factors; the presence of endogenous hyperinsulinemia combined with hypertriglyceridemia (HTG), increased body mass index, and a decreased HDL-C increase the risk of CHD death in patients with T2DM. Castro et al, 2003, Curr Hypertens Rep. 5(5):393-401; Lastra et al. 2006, Curr Diab Rep. 6(3):207-12.

### Cholesterol Transport

The fat-transport system can be divided into two pathways: an exogenous one for cholesterol and triglycerides absorbed from the intestine and an endogenous one for cholesterol and triglycerides entering the bloodstream from the liver and other non-hepatic tissue.

In the exogenous pathway, dietary fats are packaged into lipoprotein particles called chylomicrons, which enter the bloodstream and deliver their triglycerides to adipose tissue for storage and to muscle for oxidation to supply energy. The remnant of the chylomicron, which contains cholesteryl esters, is removed from the circulation by a specific receptor found only on liver cells. This cholesterol then becomes available again for cellular metabolism or for recycling to extrahepatic tissues as plasma lipoproteins.

In the endogenous pathway, the liver secretes a large, very-low-density lipoprotein particle (VLDL) into the bloodstream. The core of VLDL consists mostly of triglycerides synthesized in the liver, with a smaller amount of cholesteryl esters either synthesized in the liver or recycled from chylomicrons. Two predominant proteins are displayed on the surface of VLDL, apolipoprotein B-100 (apo B-100) and apolipoprotein E (apo E), although other apolipoproteins are present, such as apolipoprotein CIII (apo CIII) and apolipoprotein CII (apo CII). When VLDL reaches the capillaries of adipose tissue or of muscle, its triglyceride is extracted. This results in the formation of a new kind of particle called intermediate-density lipoprotein (IDL) or VLDL remnant, decreased in size and enriched in cholesteryl esters relative to a VLDL, but retaining its two apoproteins.

In human beings, about half of the IDL particles are removed from the circulation quickly, generally within two to six hours of their formation. This is because IDL particles bind tightly to liver cells, which extract IDL cholesterol to make new VLDL and bile acids. The IDL not taken up by the liver is catabolized by the hepatic lipase, an enzyme bound to the proteoglycan on liver cells. Apo E dissociates from IDL as it is transformed to LDL. Apo B-100 is the sole protein of LDL.

Primarily, the liver takes up and degrades circulating cholesterol to bile acids, which are the end products of cholesterol metabolism. The uptake of cholesterol-containing particles is mediated by LDL receptors, which are present in high concentrations on hepatocytes. The LDL receptor binds both apo E and apo B-100 and is responsible for binding and removing both IDL and LDL from the circulation. In addition, remnant receptors are responsible for clearing chylomicrons and VLDL remnants, i.e., IDL. However, the affinity of apo E for the LDL receptor is greater than that of apo B-100. As a result, the LDL particles have a much longer circulating life span than IDL particles; LDL circulates for an average of two and a half days before binding to the LDL receptors in the liver and other tissues. High serum levels of LDL are positively associated with coronary heart disease. For example, in atherosclerosis, cholesterol derived from circulating LDL accumulates in the walls of arteries. This accumulation forms bulky plaques that inhibit the flow of blood until a clot eventually forms, obstructing an artery which may ultimately lead to heart attack or stroke.

Ultimately, the amount of intracellular cholesterol liberated from the LDL controls cellular cholesterol metabolism. The accumulation of cellular cholesterol derived from VLDL and LDL controls three processes. First, it reduces the ability of the cell to make its own cholesterol by turning off the synthesis of HMGCoA reductase, a key enzyme in the cholesterol biosynthetic pathway. Second, the incoming LDL-derived cholesterol promotes storage of cholesterol by the action of cholesterol acyltransferase (“ACAT”), the cellular enzyme that converts cholesterol into cholesteryl esters that are deposited in storage droplets. Third, the accumulation of cholesterol within the cell drives a feedback mechanism that inhibits cellular synthesis of new LDL receptors. Cells, therefore, adjust their complement of LDL receptors so that enough cholesterol is brought in to meet their metabolic needs, without overloading.

High levels of apo B-containing lipoproteins can be trapped in the subendothelial space of an artery and undergo oxidation. The oxidized lipoprotein is recognized by scavenger receptors on macrophages. Binding of oxidized lipoprotein to the scavenger receptors can enrich the macrophages with cholesterol and cholesteryl esters independently of the LDL receptor. Macrophages can also produce cholesteryl esters by the action of ACAT. LDL can also be complexed to a high molecular weight glycoprotein called apolipoprotein(a), also known as apo(a), through a disulfide bridge. The LDL-apo(a) complex is known as Lipoprotein(a) or Lp(a). Elevated levels of Lp(a) are detrimental, having been associated with atherosclerosis, coronary heart disease, myocardial infarction, stroke, cerebral infarction, and restenosis following angioplasty. Wang et al. 2006, J Lipid Res. 5.

### Reverse Cholesterol Transport

Peripheral (non-hepatic) cells predominantly obtain their cholesterol from a combination of local synthesis and uptake of preformed sterol from VLDL and LDL. Cells expressing scavenger receptors, such as macrophages and smooth muscle cells, can also obtain cholesterol from oxidized apo B-containing lipoproteins. In contrast, reverse cholesterol transport (RCT) is the pathway by which peripheral cell cholesterol can be returned to the liver for recycling to extrahepatic tissues, hepatic storage, or excretion into the intestine in bile. The RCT pathway represents the only means of eliminating cholesterol from most extrahepatic tissues and is crucial to the maintenance of the structure and function of most cells in the body.

The enzyme in blood involved in the RCT pathway, lecithin:cholesterol acyltransferase (LCAT), converts cell-derived cholesterol to cholesteryl esters, which are sequestered in HDL destined for removal. LCAT is produced mainly in the liver and circulates in plasma associated with the HDL fraction. Cholesterol ester transfer protein (CETP) and another lipid transfer protein, phospholipid transfer protein (PLTP), contribute to further remodeling the circulating HDL population. PLTP supplies lecithin to HDL, and CETP can move cholesteryl esters made by LCAT to other lipoproteins, particularly apoB-containing lipoproteins, such as VLDL. HDL triglycerides can be catabolized by the extracellular hepatic triglyceride lipase and lipoprotein cholesterol is removed by the liver via several mechanisms.

Each HDL particle contains at least one molecule, and usually two to four molecules, of apolipoprotein A I (apo A I). Apo A I is synthesized by the liver and small intestine as preproapolipoprotein, which is secreted as a proprotein that is rapidly cleaved to generate a mature polypeptide having 243 amino acid residues. Apo A I consists mainly of a 22 amino acid repeating segment, spaced with helix-breaking proline residues. Apo A I forms three types of stable structures with lipids: small, lipid-poor complexes referred to as pre-beta-1 HDL; flattened discoidal particles, referred to as pre-beta-2 HDL, which contain only polar lipids (e.g., phospholipid and cholesterol); and spherical particles containing both polar and nonpolar lipids, referred to as spherical or mature HDL (HDL3 and HDL2). Most HDL in the circulating population contains both apo A I and apo A II, a second major HDL protein. The apo A I- and apo A II-containing fraction is referred to herein as the AI/AII-HDL fraction of HDL. The fraction of HDL containing only apo A I, referred to herein as the AI HDL fraction, appears to be more effective in RCT. Certain epidemiologic studies support the hypothesis that the Al-HDL fraction is antiartherogenic. Spady et al. 1999, Circulation. 100:576-578; Fielding C J, Fielding P E. 1995, J Lipid Res. 36:211-228.

The LCAT reaction requires an apolipoprotein such as apo A I or apo A-IV as an activator. ApoA-I is one of the natural cofactors for LCAT. The conversion of cholesterol to its HDL-sequestered ester prevents re-entry of cholesterol into the cell, resulting in the ultimate removal of cellular cholesterol.

HDL is not only involved in the reverse transport of cholesterol, but also plays a role in the reverse transport of other lipids, e.g. the transport of lipids from cells, organs, and tissues to the liver for catabolism and excretion. Such lipids include sphingomyelin, oxidized lipids, and lysophophatidylcholine. For example, Robins and Fasulo have shown that HDL stimulates the transport of plant sterol by the liver into bile secretions. Robins and Fasulo (1997, J. Clin. Invest. 99:380 384.

### The Role of Chromium

Dietary supplementation of chromium to normal individuals has been reported to lead to improvements in glucose tolerance, serum lipid concentrations, including high-density lipoprotein cholesterol, insulin and insulin binding. Anderson, 1986 Clin. Psychol. Biochem. 4:31-41. Supplemental chromium in the trivalent form, e.g. chromic chloride, is associated with improvements of risk factors associated with adult-onset (Type 2) diabetes and cardiovascular disease.

Chromium is a nutritionally essential trace element. The essentiality of chromium in the diet was established in 1959 by Schwartz. Schwartz, “Present Knowledge in Nutrition,” page 571, fifth edition (1984, the Nutrition Foundation, Washington, DC). Chromium depletion is characterized by the disturbance of glucose, lipid and protein metabolism and by a shortened lifespan. Chromium is essential for optimal insulin activity in all known insulin-dependent systems. Boyle et al., 1977 Southern Med. J. 70:1449-1453. Insufficient dietary chromium has been linked to both maturity-onset diabetes and to cardiovascular disease.

The principal energy sources for the body are glucose and fatty acids. Chromium depletion results in biologically ineffective insulin and compromised glucose metabolism. Under these conditions, the body relies primarily upon lipid metabolism to meet its energy requirements, resulting in the production of excessive amounts of acetyl-CoA and ketone bodies. Some of the acetyl-CoA can be diverted to increased cholesterol biosynthesis, resulting in hypercholesterolemia. Diabetes mellitus is characterized in large part by glycosuria, hypercholesterolemia, and often ketoacidosis. The accelerated atherosclerotic process seen in diabetics is associated with hypercholesterolemia Boyle et al., supra.

Chromium functions as a cofactor for insulin. It binds to the insulin receptor and potentiates many, and perhaps all, of its functions. Boyle et al., supra. These functions include, but are not limited to, the regulation of carbohydrate and lipid metabolism. Present Knowledge in Nutrition, supra, at p. 573-577. The introduction of inorganic chromium compounds per se into individuals is not particularly beneficial. Chromium must be converted endogenously into an organic complex or must be consumed as a biologically active molecule. Only about 0.5% of ingested inorganic chromium, however, is assimilated into the body. Recommended Daily Allowances, Ninth Revised Edition, The National Academy of Sciences, page 160, 1980. Only 1-2% of most organic chromium compounds are assimilated into the body.

U.S. Pat. No. Re. 33,988 discloses that when selected essential metals, including chromium, are administered to mammals as exogenously synthesized coordination complexes of picolinic acid, they are directly available for absorption without competition from other metals. This patent describes a composition and method for selectively supplementing the essential metals in the human diet and for facilitating absorption of these metals by intestinal cells. These complexes are safe, inexpensive, biocompatible, and easy to produce. These exogenously synthesized essential metal coordination complexes of picolinic acid (pyridine-2-carboxylic acid) have the following structural formula:

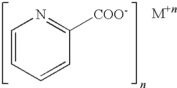

wherein M represents the metallic cation and n is equal to the cation's valence. For example, when M is Cr and n=3, then the compound is chromic tripicolinate. Other chromium picolinates disclosed include chromic monopicolinate and chromic dipicolinate.

The U.S. Recommended Daily Intake (RDI) of chromium is 120 μg. U.S. Pat. No. 5,087,623, the entire contents of which are hereby expressly incorporated herein by reference, describes the administration of chromic tripicolinate for the treatment of adult-onset diabetes in doses ranging from 50 to 500 μg. U.S. Pat. No. 6,329,361, the entire contents of which are hereby expressly incorporated herein by reference, discloses the use of high doses of chromic tripicolinate (providing 1,000-10,000 μg chromium/day) for reducing hyperglycemia and stabilizing the level of serum glucose in humans with Type 2 diabetes. U.S. Pat. Nos. 5,789,401 and 5,929,066, the entire contents of which are hereby expressly incorporated herein by reference, disclose a chromic tripicolinate-biotin composition and its use in lowering blood glucose levels in humans with Type 2 diabetes.

U.S. Pat. Nos. 5,087,623; 5,087,624; and 5,175,156, the entire contents of which are hereby expressly incorporated herein by reference, disclose the use of chromium tripicolinate for supplementing dietary chromium, reducing hyperglycemia and stabilizing serum glucose, increasing lean body mass and reducing body fat, and controlling serum lipid levels, including the lowering of undesirably high serum LDL-cholesterol levels and the raising of serum High Density Lipid (HDL)-cholesterol levels. U.S. Pat. Nos. 4,954,492 and 5,194,615, the entire contents of which are hereby expressly incorporated by reference, describe a related complex, chromic nicotinate, which is also used for supplementing dietary chromium and lowering serum lipid levels. Picolinic acid and nicotinic acid are position isomers having the following structures:

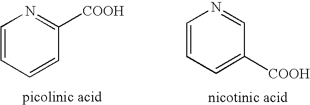

Nicotinic acid and picolinic acid form coordination complexes with monovalent, divalent and trivalent metal ions and facilitate the absorption of these metals by transporting them across intestinal cells and into the bloodstream. Chromium absorption in rats following oral administration of CrCl₃ was facilitated by the non-steroidal anti-inflammatory drugs (NSAIDs) aspirin and indomethacin. Davis et al., 1995, J. Nutrition Res. 15:202-210 (1995); Kamath et al., 1997, J. Nutrition 127:478-482. These drugs inhibit the enzyme cyclooxygenase which converts arachidonic acid to various prostaglandins, resulting in inhibition of intestinal mucus formation and lowering of intestinal pH which facilitates chromium absorption.

U.S. Pat. No. 4,315,927 teaches that when selected essential metals are administered to mammals as exogenously synthesized coordination complexes of picolinic acid, they are directly available for absorption without competition from other metals. These complexes are safe, inexpensive, biocompatible and easy to produce.

There remains a need for sources of chromium that exhibit favorable absorption profiles, and also that provide for the release of chromium from the coordination complex once within the cell.

### SUMMARY OF THE INVENTION

Provided herein are compositions comprising chromium and histidine, chromium histidinate, chromium histidinate complexes, and combinations thereof, e.g. chromium with histidinate or histidinate complex or poly histidinate or mono histidinate. In certain embodiments, the compositions described herein can be used in combination with other therapeutics, such as hypocholesterolemic and hypoglycemic therapeutic agents.

Some embodiments relate to pharmaceutical compositions comprising one or more compositions disclosed herein, with a pharmaceutically acceptable vehicle, excipient, or diluent. For example, pharmaceutically acceptable vehicles can include carriers, excipients, diluents, and the like, as well as combinations or mixtures thereof.

The compositions disclosed herein provide unexpected benefits over different sources of chromium, including various known chromium complexes, in the treatment and prevention a variety of diseases and conditions in which chromium supplementation is beneficial, such as, but not limited to, cardiometabolic syndrome, aging, Alzheimer's Disease, cancer, cardiovascular disease, diabetic nephropathy, diabetic retinopathy, disorders of glucose metabolism, disorders of lipid metabolism, dyslipidemia, dyslipoproteinemia, hypertension, impotence, inflammation, insulin resistance, obesity, pancreatitis, Parkinson's disease, peroxisome proliferator activated receptor-associated disorders, renal disease, septicemia, Syndrome X, and thrombotic disorder. Compounds and methods of the invention can also be used to modulate C-reactive protein, enhance bile production, and eliminate lipids, phospholipids, and oxysterols in bile in subjects.

Accordingly, provided herein are methods of treating or preventing cardiometabolic syndrome or a condition associated therewith in a subject that has been identified as having, or identified as being at risk of developing, CMS or a condition associated therewith, by providing said subject a composition that contains chromium and histidine, chromium histidinate complexes, or combinations thereof alone or in combination with at least one other chromium complex in combination with chromium histidinate.

Also provided herein are methods for inhibiting hepatic fatty acid and sterol synthesis in subjects in need thereof, by identifying subjects in need of inhibition of hepatic fatty acids or inhibition of sterol, and providing a therapeutically effective amount of a composition disclosed herein to the subject.

Also provided are methods for increasing HDL levels in a subject in need of increased HDL levels, by identifying a subject in need of increased HDL levels, and providing a therapeutically effective amount of a composition disclosed herein to the subject. Accordingly, embodiments disclosed herein also relate to the treatment or prevention of diseases or disorders capable of being treated or prevented by increasing HDL levels in subjects identified as being in need thereof

Provided herein are methods for lowering LDL levels in subjects in need of a reduction in LDL levels by providing a therapeutically effective amount of a composition disclosed herein to said subject.

Further provided herein are methods of improving endothelial function in a subject in need of improved endothelial function by identifying a subject in need of improved endothelial function, e.g. by routine clinical methods, and providing a therapeutically effective amount of a therapeutically effective amount of a composition disclosed herein to said subject.

Disclosed herein are methods for improving at least one of the following: blood pressure, vascular tone, vascular relaxation, and coronary blood flow in a subject in need thereof by identifying a subject in need of improved blood pressure, vascular tone, vascular relaxation, and coronary blood flow using routine clinical methods, and providing the subject can be a therapeutically effective amount of a composition disclosed herein.

Also provided are methods for lowering fasting and post prandial blood sugar levels, lowering serum triglyceride levels and improving insulin sensitivity in a subject in need thereof by identifying a subject in need of a reduction in fasting and/or post-prandial blood sugar levels, and providing the subject a therapeutically effective amount of a composition disclosed herein.

The compositions disclosed herein can improve fasting and post prandial blood insulin levels, decrease hyperinsulinemia and decrease insulin resistance in mammals. Accordingly, some embodiments provide methods for treatment or prevention of cardiometabolic syndrome-associated disorders, such as hyperglycemia, hyperinsulinemia, or insulin resistance, by providing a therapeutically effective amount of a composition disclosed herein to a subject in need of improved fasting and post-prandial blood insulin levels, treatment for hyperinsulinemia, or a decrease in insulin resistance.

The compositions disclosed herein can decrease body fat and increase lean body mass, thereby effectuating improvements in body composition in mammals. Accordingly, some embodiments provide methods for decreasing body fat or increasing lean body mass in an subject by identifying a subject in need of a decrease in body fat or increase in lean body mass, and providing to said subject a therapeutically amount of a composition disclosed herein.

The compositions disclosed herein can decrease inflammatory markers, the risk of CVD and diabetes, and reduce obesity in mammals. Accordingly, some embodiments provide methods of decreasing inflammatory markers, decreasing the risk of CVD and diabetes, or reducing obesity in mammals. A subject in need of a decrease in inflammatory markers, a subject at risk of CVD and diabetes, or a subject that is obese can be identified, and provided a therapeutically effective amount of a composition disclosed herein.

The compositions disclosed herein can decrease markers associated with renal function disorders and improve renal function in mammals. Accordingly, provided herein are methods for the treatment or prevention of renal disorders, by identifying a subject with or at risk of developing a renal disorder, e.g., a subject with cardiometabolic syndrome and a renal disorder, and providing a therapeutically effective amount of a composition disclosed herein to said subject.

The compositions disclosed herein can decrease inflammatory markers associated with bone health and can improve bone health or treat bone disorders. Accordingly, some embodiments provide methods of treatment or prevention of arthritis and rheumatic heart disease, for example in subjects with cardiometabolic disorder. A subject can be identified as having increased inflammatory markers and administered a composition described herein. In some embodiments, the subject can be identified as having cardiometabolic syndrome, for example accompanied by arthritis and rheumatic heart disease and administered a composition described herein.

The compositions disclosed herein can improve immune function associated with cardiometabolic syndrome, for example in mammals. Accordingly, provided herein are methods for treating or preventing immune function disorders in subjects by identifying a subject with cardiometabolic syndrome and administering to the subject a therapeutically effective amount of a composition described herein.

The compositions disclosed herein can improve metabolic function associated with cardiometabolic syndrome, diabetes, obesity and cardiovascular disease, for example in mammals. Accordingly, some embodiments provide methods of improving metabolic function by identifying a subject with cardiometabolic syndrome, diabetes, obesity, or cardiovascular disease and administering a therapeutically effective amount of a composition described herein to the subject.

The compositions disclosed herein can improve chromium status associated with cardiometabolic syndrome, diabetes, obesity and cardiovascular disease. Accordingly, some embodiments provide methods of treatment or prevention of cardiometabolic syndrome disorders with low chromium status or deficiency of chromium. Some embodiments provide methods of improving chromium depletion in tissues due to chronic conditions, such as diabetes, obesity and cardiovascular disease. A subject with cardiometabolic syndrome, diabetes, obesity, or cardiovascular disease and chromium depletion can be identified and provided a therapeutically effective amount of a composition disclosed herein.

The compositions disclosed herein can improve amino acid profile status associated with cardiometabolic syndrome, diabetes, obesity and cardiovascular disease, for example in mammals. Accordingly, provided herein are methods of treatment or prevention of cardiometabolic syndrome disorders with low amino acid profiles or protein deficiencies. Also provided are methods of improving amino acid absorption in tissues due to chronic conditions such as diabetes, obesity and cardiovascular disease. Subjects with cardiometabolic syndrome, diabetes, obesity, or cardiovascular disease and low amino acid profiles or amino acid proteins deficiencies can be identified and provided a therapeutically effective amount of a composition disclosed herein

The compositions disclosed herein can improve chromium absorption and amino acid profile status associated with cardiometabolic syndrome, diabetes, obesity and cardiovascular disease in mammals and therefore the invention also encompasses methods of improving amino acid profiles, protein deficiencies, and chromium deficiencies in these individuals. Individuals with cardiometabolic syndrome, diabetes, obesity or cardiovascular disease with associated low amino acid profiles and chromium deficiencies can be identified and administered a composition disclosed herein.

The compositions disclosed herein can improve exchange and transport of amino acids, proteins and chromium in tissues associated with conditions such as cardiometabolic syndrome, diabetes, obesity and cardiovascular disease in mammals. Therefore, provided herein are methods of treatment or prevention of cardiometabolic syndrome disorders and associated disorders and methods of improving the exchange and transport of chromium and amino acid exchange for normal functions of the organs in the body. Further provided are methods for improving amino acid profile or deficiency of protein or all amino acids, methods for improving amino acid profile depletion, and methods for improving amino acid absorption due to chronic conditions and to replete the amino acids levels in tissues. Subjects with cardiometabolic syndrome, diabetes, obesity or cardiovascular disease with associated low amino acid profiles and chromium deficiencies can be identified and administered a composition disclosed herein

The compositions disclosed herein favorably alter lipid metabolism in mammals with dyslipidemia at least in part by enhancing oxidation of fatty acids through the ACC/malonyl-CoA/CPT-I regulatory axis. Accordingly, provided herein are methods of treatment or prevention of cardiometabolic syndrome disorders associated with dyslipidemia by identifying subjects with cardiometabolic syndrome and administering a composition disclosed herein to the subject.

Further embodiments provide methods for reducing the abdominal fat in a by identifying a subject in need of fat-content reduction and administering to the subject a therapeutically effective amount of a compound disclosed herein.

Also provided are methods for reducing total cholesterol, or improving cholesterol profiles in a subject in need of cholesterol reduction or an improvement in cholesterol profile. A subject with elevated cholesterol or in need of improved cholesterol profiles can be identified and administered a composition disclosed herein.

### BRIEF DESCRIPTION OF THE DRAWINGS

FIG. 1 is a bar graph depicting levels of triglycerides secreted into culture media by cell cultures treated with the indicated amounts of chromium histidinate, in the presence or absence of insulin, as indicated.

FIG. 2 is a graph depicting levels of glucose in media of cells cultured in the presence of the indicated amounts of chromium histidinate, in the presence or absence of insulin, as indicated.

FIG. 3 is a bar graph showing the glucose levels in normal rats fed a standard diet, with or without supplementation with chromium histidinate.

FIG. 4 is a bar graph showing the difference in insulin levels in normal rats fed a standard diet, with or without supplementation with chromium histidinate.

FIG. 5 is a bar graph showing the difference in insulin sensitivity levels in normal rats fed a standard diet, with or without supplementation with chromium histidinate.

FIG. 6 is s bar graph showing the difference in total cholesterol levels in normal rats fed a standard diet, with or without supplementation with chromium histidinate.

FIG. 7 is s bar graph showing the difference in triglyceride levels in normal rats fed a standard diet, with or without supplementation with chromium histidinate.

FIG. 8 is a bar graph showing the difference in free fatty acid levels in normal rats fed a standard diet, with or without supplementation with chromium histidinate.

FIG. 9 is a bar graph showing the difference in serum chromium levels normal rats fed a standard diet, with or without supplementation with chromium histidinate.

FIG. 10 is a bar graph showing the difference in blood glucose levels in fat “insulin resistant” rats fed a high fat diet, with or without supplementation with chromium histidinate.

FIG. 11 is a bar graph showing the difference in insulin levels in fat “insulin resistant” rats fed a high fat diet, with or without supplementation with chromium histidinate.

FIG. 12 is a bar graph showing the difference in insulin sensitivity in fat “insulin resistant” rats fed a high fat diet, with or without supplementation with chromium histidinate.

FIG. 13 is a bar graph showing the difference in total cholesterol levels in fat “insulin resistant” rats fed a high fat diet, with or without supplementation with chromium histidinate.

FIG. 14 is a bar graph showing the difference in triglyceride levels in fat “insulin resistant” rats fed a high fat diet, with or without supplementation with chromium histidinate.

FIG. 15 is a bar graph showing the difference in free fatty acid levels in fat “insulin resistant” rats fed a high fat diet, with or without supplementation with chromium histidinate.

FIG. 16 is a bar graph showing the difference in body weight in fat “insulin resistant” rats fed a high fat diet, with or without supplementation with chromium histidinate.

FIG. 17 is a bar graph showing the difference cortisol levels in fat “insulin resistant” rats fed a high fat diet, with or without supplementation with chromium histidinate.

FIG. 18 is a bar graph showing the difference in blood glucose levels in rats fed a high fat diet and treated with streptozotocin, with or without supplementation with chromium histidinate.

FIG. 19 is a bar graph showing the difference in insulin levels in rats fed a high fat diet and treated with streptozotocin, with or without supplementation with chromium histidinate.

FIG. 20 is a bar graph showing the difference in insulin sensitivity in rats fed a high fat diet and treated with streptozotocin, with or without supplementation with chromium histidinate.

FIG. 21 is a bar graph showing the difference in total cholesterol levels in rats fed a high fat diet and treated with streptozotocin, with or without supplementation with chromium histidinate.

FIG. 22 is a bar graph showing the difference in triglyceride levels in rats fed a high fat diet and treated with streptozotocin, with or without supplementation with chromium histidinate.

FIG. 23 is a bar graph showing the difference in free fatty acid levels in rats fed a high fat diet and treated with streptozotocin, with or without supplementation with chromium histidinate.

FIG. 24 is a bar graph showing the difference in serum chromium levels in rats fed a high fat diet and treated with streptozotocin, with or without supplementation with chromium histidinate.

FIG. 25 is a bar graph showing the difference in cortisol levels in rats fed a high fat diet and treated with streptozotocin, with or without supplementation with chromium histidinate.

### DETAILED DESCRIPTION OF THE PREFERRED EMBODIMENT

Embodiments disclosed herein relate to the use of compositions comprising, consisting essentially of, or consisting of chromium and histidine, chromium histidinate complex, chromium trihistidinate, or chromium poly histidinate complex, or combinations thereof, including pharmaceutically acceptable salts, hydrates, solvates, or mixtures thereof for the treatment of cardiometabolic syndrome and related conditions, diseases, and disorders.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the invention herein described.

### The Role of Histidine/Histidinate

Histidine is one of the 20 most common natural amino acids present in proteins. In the nutritional sense, in humans, hisitidine is considered an essential amino acid for normal healthy function. The imidazole side chains and the relatively neutral pKa of histidine (ca 6.0) mean that relatively small shifts in cellular pH will change its charge. For this reason, this amino acid side chain finds its way into considerable use as a coordinating ligand in metalloproteins, and also as a catalytic site in certain enzymes. The imidazole side chain has two nitrogens with different properties: one is bound to hydrogen and donates its lone pair to the aromatic ring and as such is slighty acidic; the other one donates only one electron to the ring so it has a free lone pair and is basic. These properties are exploited in different ways in proteins. In catalytic triads, the basic nitrogen of histidine is used to abstract a proton from serine, threonine or cysteine to activate it as a nucleophile. In a histidine proton shuttle, histidine is used to quickly shuttle protons, it can do this by abstracting a proton with its basic nitrogen to make a positively-charged intermediate and then use another molecule, a buffer, to extract the proton from its acidic nitrogen. In carbonic anhydrases, a histidine proton shuttle is utilized to rapidly shuttle protons away from a zinc-bound water molecule to quickly regenerate the active form of the enzyme. The amino acid is a precursor for histamine and carnosine biosynthesis.

Histidine has two enantiomeric forms: D-histidine and L-histidine. The structure of histidine is shown below. Histidine is a basic, essential amino acid that is also a precursor of histamine, a compound released by immune system cells during an allergic reaction. Histamine is needed for growth and for the repair of tissue, as well as the maintenance of the myelin sheaths that act as protector for nerve cells. It is further required for the manufacture of both red and white blood cells, and helps to protect the body from damage caused by radiation and in removing heavy metals from the body. In the stomach, histidine is also helpful in producing gastric juices, and people with a shortage of gastric juices or suffering from indigestion, may also benefit from this nutrient. Histidine is also used for sexual arousal, functioning and enjoyment. Histidinemia is an inborn error of the metabolism of histidine due to a deficiency of the enzyme histidase, where high levels of histidine are found in the blood and urine, and may manifest in speech disorders and mental retardation.

Described herein are compositions that comprise, consist essentially of, or consist of chromium and histidine, or chromium histidinate complexes, such as chromium histidinate chromium trihistidinate, and chromium polyhistidinate, or combinations thereof, exhibit improved absorption in mammals over other known chromium complexes. In particular, the compositions described herein show superior absorption and intracellular release of chromium from the histidinate complex.

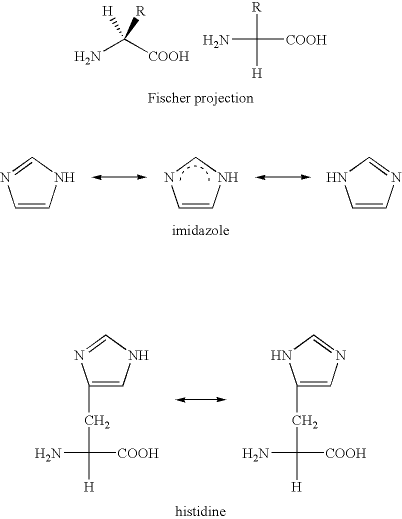

As discussed above, the compositions disclosed herein can include chromium and histidine, or chromium histidinate complexes alone or in combination with other chromium complexes including chromium picolinate, chromium nicotinate, chromium chloride, tri-chromium(III) oxo acetate cluster ([Cr(3)O(OAc)(6)](+)), biomimetic cation [Cr(3)O(O(2)CCH(2)CH(3))(6)(H(2)O)(3)](+) and chromium triphenylanine, and a chromium complex now known or discovered in the future.

The compositions described herein can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compounds' enantiomers and stereoisomers, that is, both the stereomerically pure form (e.g., geometrically pure, enantiomerically pure, or diastereomerically pure) and enantiomeric and stereoisomeric mixtures.

As used herein, a composition that “substantially” comprises a compound means that the composition contains more than about 80% by weight, more preferably more than about 90% by weight, for example 91%, 92%, 93%, 94%, 95%, 96%, 97%, 98%, 99% or more by weight.

As used herein, a composition that “substantially” comprises a chromium complex means that the composition contains more than or equal to 7.0% of trivalent or dietary chromium. In some embodiments, the composition can include a certificate of analysis that indicates certain properties of the composition, i.e., that the composition is negative for microbial growth, yeast and/or mold, and that toxic metals are less than 1 ppm.

In some embodiments, the compositions disclosed herein are in the form of pharmaceutically effective salts. The phrase “pharmaceutically acceptable salt(s),” as used herein includes, but is not limited to, salts of acidic or basic groups that may be present in the compounds disclosed herein. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds disclosed herein that include an amino moiety also can form pharmaceutically acceptable salts with various amino acids, in addition to the acids mentioned above. Compounds disclosed herein that are acidic in nature are capable of forming base salts with various pharmacologically acceptable cations. Examples of such salts include alkali metal or alkaline earth metal salts and, particularly, calcium, magnesium, sodium lithium, zinc, potassium, silicon, phosphorus and iron salts.

As used herein, the term “hydrate” means a compound disclosed herein3 or a salt thereof, that further includes a stoichiometric or non-stoichiometric amount of water bound by non-covalent intermolecular forces. The term hydrate includes solvates, which are stoichiometric or non-stoichiometric amounts of a solvent bound by non-covalent intermolecular forces. Preferred solvents are volatile, non-toxic, and/or acceptable for administration to humans in trace amounts.

In accordance with the methods disclosed herein, the effective dose of chromium provided by the chromium complex can be at least 50 μg per day, for example at least 60 μg, at least 70 μg, at least 80 μg, at least 90 μg, at least 100 μg, at least 125 μg, at least 150 μg, at least 200 μg, at least 250 μg, at least 300 μg, at least 350 μg, at least 400 μg, at least 450 μg, at least 500 μg, at least 550 μg, at least 600 μg, at least 650 μg, at least 700 μg, at least 750 μg, at least 800 μg, at least 850 μg, at least 900 μg, at least 950 μg, at least 1,000 g, at least 1500 μg, at least 2,000 μg, at least 2500 μg, at least 3000 μg, at least 3500 μg, at least 4000 μg, at least 4500 μg or at least 5000 μg chromium complex/day. The chromium complex can be a trivalent chromium complex such as chromium picolinate, chromic tripicolinate, chromium nicotinate, chromic polynicotinate, chromium chloride, chromium histidinate, chromium yeast, or any other chromium complex, whether now known or to be developed in the future, or any combination thereof

By way of example, the level of chromium used for supplementation in order to inhibit the onset of insulin resistance is at least about 50 μg/day. Note in particular that chromium picolinate and chromium chloride have been administered to rats at levels several thousand times the upper limit of the estimated safe and adequate daily dietary intake (ESADDI) for chromium for humans (based on body weight) without toxic effects. R. Anderson et al., Lack of Toxicity of Chromium Chloride and Picolinate, 16 J. Am. Coll. Nutr. 273-279 (1997). While the level of chromium used for supplementation can be within several thousand times the upper limit of the ESADDI, preferably, the amount of chromium is between about 50 and 2,000 μg/day. For example, the amount of chromium can be between about 300 and 1,000 μg/day, e.g. between about 400 and 1,000 μg/day (e.g. 500, 600, 700, 800, 900, or 1,000 μg/day, or any number in between). In some embodiments, the amount of chromium is between about 600 and 1,000 μg/day. Note that these doses are based on a 70 kg adult human, and that the dose can be applied on a per-kilogram basis to humans or animals of different weights.

In some embodiments, the chromium complex can be in a pharmaceutically acceptable carrier.

Optionally, the chromium complex is orally administered. However, in some aspects of the invention, the chromium complex is parenterally administered, or administered by any other route, such as transdermally or the like.

In some embodiments, certain chelating agents can be added to facilitate absorption of the chromium complex. Optionally, the ratio of the chromium complex to the chelating agent is between about 10:1 to about 1:10 (w/w), e.g. 10:1, 10:2, 10:3, 10:4, 10:5, 10:6, 10:7, 10:8, 10:9, 1:1, 1:2, 1:3, 1:4, 1:5, 1:6, 1:7, 1:8, 1:9, 1:10, or any number in between. In one aspect of the invention, picolinic acid is administered to an individual. In another aspect, nicotinic acid is administered to an individual. In still another aspect, both picolinic and nicotinic acid are administered to an individual in order to inhibit the onset of drug-insulin resistance.

In some embodiments, the compositions disclosed herein are provided in an amount effective for the prevention of insulin resistance. As used herein, the term “insulin resistance”, or “(IR)” refers to a physiologically abnormal state in which cells do not respond appropriately to insulin, such that glucose in the blood cannot efficiently enter cells and, therefore, leads to hyperglycemia. The cardiovascular and metabolic disturbances associated with IR can individually and interdependently lead to a substantial increase in cardiovascular disease (CVD) morbidity and mortality, making the cardiometabolic syndrome an established and strong risk factor for premature and severe CVD and stroke. In some embodiments provided herein, a subject is provided a composition comprising chromium histidinate alone or in combination with a sufficient amount of a chromium complex to inhibit IR or reduce the risk of the onset of IR. The chromium complex can include chromium picolinate, chromic tripicolinate, chromium nicotinate, chromic polynicotinate, chromium chloride, chromium histidinate, chromium yeast, or other chromium complex, whether now known or to be developed in the future. In some embodiments, the amount of chromium provided by the chromium complex and contained in the composition is between about 50 μg and 2000 μg, as discussed above.

Advantageously, an individual is administered a pharmaceutically effective dose of a chromium complex such as chromium histidinate alone or in combination with at least one other chromium complex. In one embodiment, a composition disclosed herein (e.g., chromium histidinate) and another chromium complex are administered substantially simultaneously. In an alternative embodiment, the compositions disclosed herein (e.g., chromium histidinate) and another chromium complex are provided to the subject sequentially in either order. If administered separately, the chromium complex and diet and composition disclosed herein (e.g., chromium histidinate) should be given in a temporally proximate manner, e.g., within a twenty-four hour period. More particularly, the chromium complex and composition disclosed herein (e.g., chromium histidinate) can be given within one hour of each other.

One of skill in the art will appreciate that other components (e.g., foods, beverages, bars, or the like) can be added to the compositions described herein separately or incorporated into a single formulation to enhance the effects of chromium. As will be described in greater detail below, uncomplexed chelating agents such as nicotinic acid, picolinic acid, or both nicotinic and picolinic acids can be included in the formulation or added separately to enhance the absorption of the chromium complex.

In some embodiments, the chromium complexes described herein can be administered with a food, beverage, bar, or the like which induces insulin resistance. In some embodiments, the chromium complex is administered first and then a food, beverage or bars which induce insulin resistance is administered second. In yet another embodiment, a food, beverage, or bar which induces insulin resistance is administered first. If administered separately, the chromium complex and the food, beverage, or bar which induces insulin resistance can be given in a temporally proximate manner, e.g. within a twenty-four hour period, such that the inhibition of functional foods/beverages or bars-induced insulin resistance is enhanced. More particularly, the chromium complex and food, beverage, bar, or the like which induces insulin resistance can be given within one hour of each other. In some embodiments, the food, beverage, bar or the like which induces insulin resistance can be prepared as a single formulation to include both the functional food, beverage, bar, or the like and an effective dose of a chromium complex. One of skill in the art will appreciate that other components can be added separately or incorporated into a single formulation to enhance the effects of chromium in inhibiting food or beverage-induced insulin resistance.

In some embodiments, the chromium complexes described herein can be provided with a drug which induces IR. In some embodiments, the chromium complex can administered first and then the drug which induces insulin resistance is added second. In some embodiments, the drug which induces insulin resistance is administered first. If administered separately, the chromium complex and drug which induces insulin resistance can be given in a temporally proximate manner, e.g. within a twenty-four hour period, such that the inhibition of drug-induced insulin resistance is enhanced. For example, the chromium complex and drug which induces insulin resistance can be given within one hour of each other. In one embodiment, the drug which induces insulin resistance is prepared as a single formulation to include both the active ingredient of the drug and an effective dose of a chromium complex. One of skill in the art will appreciate that other components can be added separately or incorporated into a single formulation to enhance the effects of chromium in inhibiting drug-induced insulin resistance. As will be described in greater detail below, uncomplexed chelating agents such as nicotinic acid, picolinic acid, or both nicotinic and picolinic acids can be included in the formulation or added separately to enhance the absorption of the chromium complex.

While the chromium complexes aid in the absorption of chromium by intestinal cells, in some embodiments, uncomplexed chelating agents are advantageously included in the compositions to facilitate absorption of other ingested chromium as well as other metals including, but not limited to, copper, iron, magnesium, manganese, and zinc. Suitable chelating agents include histidine, any essential amino D or L amino acids, tri amino acid formulae including but not limited to, triphenylalanine, tri histidine, tri arginine, picolinic acid, nicotinic acid, or both picolinic acid and nicotinic acid. Thus, the compositions of the disclosed invention are readily absorbable forms of chromium complex which also facilitate absorption of other essential metals in the human diet.

Chelating agents such as histidine, picolinic acid and nicotinic acid are available from many commercial sources, including Sigma-Aldrich (St. Louis, MO) (picolinic acid; catalog No. P5503; nicotinic acid; catalog No. PN4126). In some embodiments, the ratio of the chromium complex to the chelating agent from about 10:1 to about 1:10 (w/w), more preferably from about 5:1 to about 1:5 (w/w), e.g. 5:1, 5:2, 5:3, 5:4, 1:1; 1:2, 1:3, 1:4, 1:5, or any number in between. Alternatively, the molar ratio of chromium complex to the uncomplexed chelating agent is preferably 1:1, and can be from about 5:1 to about 1:10, e.g. e.g. 5:1, 5:2, 5:3, 5:4, 1:1; 1:2, 1:3, 1:4, 1:5, 1:6, 1:7, 1:8, 1:9, 1:10, or any number in between. The chelating agents with D or L amino acid and or with tri or mono and di forms of chromium complex with tri amino acid or one or more amino acids but not limited to chromium triphenylanine, chromium trihistidine, chromium poly phenylanine, chromium poly hisitidine, chromium polynicotinate, chromium di phenylananine, chromium di picolinic acid, chromium di hisitidine etc.

The administration of chromium can be by any of the methods of administration described below or by drug delivery methods known by one of skill in the art. The compositions can be administered orally, through parenteral nutrition, e.g., feeding tube or intravenously, and through other known means. Chromium histidine alone or in combination with other essential nutrients but not limited to fatty acids, carbohydrates, minerals and vitamins etc. is particularly preferred as the source of chromium supplementation due to its high level of bioavailability, but any form of dietary chromium can be used in the compositions and methods described herein.

For oral administration, the chromium complex can be provided as a tablet, aqueous or oil suspension, dispersible powder or granule, emulsion, hard or soft capsule, syrup, elixir, or beverage. Compositions intended for oral use can be prepared according to any method known in the art for the manufacture of pharmaceutically acceptable compositions and such compositions can contain one or more of the following agents: sweeteners, flavoring agents, coloring agents and preservatives. Sweetening and flavoring agents can be used to increase the palatability of the preparation.

Some embodiments provide tablets containing chromium complex in admixture with non-toxic pharmaceutically acceptable excipients suitable for tablet manufacture. Pharmaceutically acceptable excipients refer to agents that compatible with the other ingredients of the formulation as well as non-injurious to the patient. Such excipients include but are not limited to inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets can be uncoated or can be coated by known techniques to delay disintegration and absorption in the gastrointestinal tract and thereby provide a sustained action over a longer period of time, for example to provide a controlled, sustained, or delayed release tablet. For example, a time delay material such as glyceryl monostearate or glyceryl distearate alone or with a wax can be employed.

Formulations comprising the compounds disclosed herein for oral use can also be presented as hard gelatin capsules wherein the active ingredient is mixed with an inert solid diluent. Non limiting examples of inert solid diluents include calcium carbonate, calcium phosphate or kaolin. In some embodiments, formulations comprising the compounds disclosed herein can be presented as soft gelatin capsules wherein the active ingredient is mixed with water or an oil medium, such as peanut oil, liquid paraffin or olive oil. In some embodiments, the compositions that contain the chromium complexes described herein can be provided in an aqueous suspensions, e.g., in admixture with excipients suitable for the manufacture of aqueous suspensions. Non-limiting examples of excipients suitable for the manufacture of aqueous suspensions include suspending agents, dispersing or wetting agents, one or more preservatives, one or more coloring agents, one or more flavoring agents and one or more sweetening agents such as sucrose or saccharin.

In some embodiments, the compounds disclosed herein can be provided in oil suspensions. Oil suspensions can be formulated by suspending the active ingredient in a vegetable oil, such as arachis oil, olive oil, sesame oil or coconut oil, or in a mineral oil such as liquid paraffin. The oil suspension can contain a thickening agent, such as beeswax, hard paraffin or cetyl alcohol, or the like. Sweetening agents, such as those set forth above, and flavoring agents can be added to provide a palatable oral preparation. These compositions can be preserved by an added antioxidant such as ascorbic acid. Dispersible powders and granules of the invention suitable for preparation of an aqueous suspension by the addition of water can be used to provide the active ingredient in admixture with a dispersing or wetting agent, a suspending agent, and one or more preservatives. Additional excipients, for example additional sweetening, flavoring and coloring agents, can also be present in the oil suspensions.

In some embodiments, the compounds described herein can be provided in a syrup or elixir. Syrups and elixirs can be formulated with sweetening agents, such as glycerol, sorbitol or sucrose or the like. In some embodiments, the syrups or elixirs can include a demulcent, a preservative, a flavoring or a coloring agent.

In some embodiments, the compounds disclosed herein are provided in a preparation for parenteral administration, e.g. in the form of a sterile injectable preparation, such as a sterile injectable aqueous or oleaginous suspension. Injectable aqueous or oleaginous suspensions can formulated according to methods well known in the art using suitable dispersing or wetting agents and suspending agents. The sterile injectable preparation can also be a sterile injectable solution or suspension in a non-toxic parenterally-acceptable diluent or solvent, such as a solution in 1,3-butanediol or the like. Non-limiting examples of suitable diluents include water, Ringer's solution, isotonic sodium chloride solution and the like. In addition, sterile fixed oils can be employed conventionally as a solvent or suspending medium. For this purpose, any bland fixed oil can be employed, such as synthetic mono or diglycerides or the like. In addition, fatty acids such as oleic acid can likewise be used in the preparation of injectable preparations.

In some embodiments, the compositions described herein can be in the form of oil-in-water emulsions. The oily phase can be a vegetable oil, such as olive oil or arachis oil, a mineral oil such as liquid paraffin, or a mixture thereof Non-limiting examples of suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. In some embodiments, the oil-in-water emulsions can contain sweetening and flavoring agents.

It will be appreciated by the skilled artisan that the amount of chromium histidine alone or in combination with chromium complex that can be combined with a carrier material to produce a single dosage form will vary depending upon the host treated and the particular mode of administration.

For example, in some embodiments, the chromium complexes can be provided in a ratio that is effective for glucose and lipid metabolism in the body of a mammal. In some embodiments, chromium histidinate alone or in combination with other chromium complexes can be provided in an amount effective for the management of glucose and lipid metabolism in the body of a mammal, e.g, between a ratio of about 0.0001 to 1000 and about 1000:0.001/kg body weight.

When administered to a mammal, e.g., to an animal for veterinary use or for improvement of livestock, or to a human for clinical use, the compounds of the invention can be administered in isolated form or as the isolated form in a pharmaceutical composition. As used herein, “isolated” means that the compounds of the invention are separated from other components of either (a) a natural source, such as a plant or cell or food, preferably bacterial culture, or (b) a synthetic organic chemical reaction mixture. In some embodiments, the compounds disclosed herein are purified. As used herein, “purified” means that when isolated, the isolate contains at least about 95% of the compound, and preferably at least 98% of the compound.

In some embodiments, the compositions disclosed herein, are provided to the subject orally. In some embodiments, the compositions disclosed herein are administered to the subjects by other routes, e.g. by intravenous infusion or bolus injection, by absorption through epithelial or mucocutaneous linings (e.g., oral mucosa, rectal and intestinal mucosa, etc.). In some embodiments, the compounds or compositions described herein can be administered together with another biologically active agent. Administration can be systemic or local. Various delivery systems useful in the methods disclosed herein are include for example, encapsulation in liposomes, microparticles, microcapsules, capsules, etc., and can be used to administer a compound of the invention. In certain embodiments, more than one composition disclosed herein is administered to a patient.

Other modes of administration useful in the methods include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In some embodiments, the mode of administration is left to the discretion of a practitioner, and will depend in part upon the site of the medical condition. In some embodiments, administration will result in the release of the compounds of the invention into the bloodstream.

In some embodiments, it can be desirable to administer one or more compounds of the invention locally to the area in need of treatment. This can be achieved, for example, and not by way of limitation, by local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers. In one embodiment, administration can be by direct injection at the site (or former site) of an atherosclerotic plaque tissue

In certain embodiments, for example, for the treatment of Alzheimer's disease, it can be desirable to introduce one or more compounds of the invention into the central nervous system by any suitable route, including intraventricular, intrathecal or epidural injection. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Notably, the disclosed compositions are useful as a nutritional supplement for achieving the disclosed effect and methods of using the same. The phrase “pharmaceutically acceptable” is intended to be interpreted in the broadest sense to include nutritional supplements, which do not require approval by a regulatory agency of the Federal or state government. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a patient, the compounds and compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use.

Compounds and compositions of the invention for oral delivery can be in the form of tablets, lozenges, aqueous or oily suspensions, granules, powders, emulsions, capsules, syrups, or elixirs. Compounds and compositions of the invention for oral delivery can also be formulated in foods and food mixes. Orally administered compositions can contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compounds and compositions of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a compound of the invention that will be effective in the treatment of a particular disorder or condition disclosed herein will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each circumstances. However, suitable dosage ranges for oral administration are generally about 0.001 milligram to 5000 milligrams of a compound of the invention per kilogram body weight. In specific preferred embodiments of the invention, the oral dose is 0.01 milligram to 1000 milligrams per kilogram body weight, more preferably 0.1 milligram to 100 milligrams per kilogram body weight, more preferably 0.5 milligram to 25 milligrams per kilogram body weight, and yet more preferably 1 milligram to 10 milligrams per kilogram body weight. The dosage amounts described herein refer to total amounts administered; that is, if more than one compound of the invention is administered, the preferred dosages correspond to the total amount of the compounds of the invention administered. Oral compositions preferably contain 10% to 95% active ingredient.

The compositions disclosed herein can preferably used as a slow acting agent or long acting agent in addition to drugs or alone before meals and or after meals. Effective doses can be extrapolated from dose-response curves derived from in vitro or animal model test systems. Such animal models and systems are well known in the art.

In some embodiments, the compositions described herein can be in the form of nutraceutical packs not limited to functional foods, beverages, bars, dietary supplements, capsules, powder form or gelatin form, pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a compound of the invention and another lipid-mediating compound, glycemic control and antihypertensive drugs, including but not limited to insulin, statin, a thiazolidinedione, or a fibrate or dietary modifications.

The compositions disclosed herein can be assayed in vitro and in vivo, for the desired therapeutic or prophylactic activity, prior to use in humans. For example, in vitro assays can be used to determine whether administration of a specific compound of the invention or a combination of compounds of the invention is preferred for lowering fatty acid synthesis. The compositions disclosed herein also can be demonstrated to be effective and safe using animal model systems.

### Therapeutic Uses of Chromium Histidine/Histidinate

In accordance with the methods disclosed herein, a composition comprising, consisting essentially of, or consisting of a chromium and histidine, chromium histidinate complex, chromium trihistidinate, or chromium polyhistidinate complex, or any combination thereof, can be provided to a subject, such as a mammal, with or at risk of developing Alzheimer's Disease, cancer, cardiovascular disease, diabetic nephropathy, diabetic retinopathy, a disorder of glucose metabolism, dyslipidemia, dyslipoproteinemia, hypertension, impotence, inflammation, insulin resistance, obesity, oxysterol elimination in bile, pancreatitis, Parkinson's disease, a peroxisome proliferator activated receptor-associated disorder, renal disease, septicemia, metabolic syndrome disorders (e.g., Syndrome X), a thrombotic disorder, a gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain, polymyositis/polymyalgia rheumatica/fibrositis, infection and arthritis, juvenile rheumatoid arthritis, tendonitis, bursitis and other soft tissue rheumatism. Also in accordance to the methods disclosed herein, the compositions described herein can be provided to a subject to treat disorders or symptoms associated with ageing, to enhance bile production, to enhance reverse lipid transport, to promote lipid elimination in bile, to modulate C reactive protein, or to enhance phospholipid elimination in bile.

As used herein, the term “treatment” or “treating” refers to an amelioration of a disease or disorder, or at least one discernible symptom thereof The term “treatment” or “treating” refers to inhibiting the progression of a disease or disorder, either physically, e.g. stabilization of a discernible symptom, or physiologically, e.g. stabilization of a physical parameter, or both.

In certain embodiments, the compounds of the invention or the compositions of the invention are provided to a subject, such as a mammal, as a preventative measure against such diseases. As used herein, “prevention” or “preventing” refers to a reduction of the risk of acquiring a given disease or disorder alone or in combination with another condition.

In some embodiments, the compositions disclosed herein are provided as a preventative measure to a patient, preferably a human having a genetic predisposition to Alzheimer's Disease, cancer, cardiovascular disease, diabetic nephropathy, diabetic retinopathy, a disorder of glucose metabolism, dyslipidemia, dyslipoproteinemia, reduced bile production, reduced reverse lipid transport, hypertension, impotence, inflammation, insulin resistance, lipid elimination in bile, modulation of C-reactive protein, obesity, oxysterol elimination in bile, pancreatitis, Parkinson's disease, a peroxisome proliferator activated receptor-associated disorder, reduced phospholipid elimination in bile, renal disease, septicemia, metabolic syndrome disorders (e.g., Syndrome X), a thrombotic disorder, inflammatory conditions and diseases such gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain, polymyositis/polymyalgia rheumatica/fibrositis, infection and arthritis, juvenile rheumatoid arthritis, tendonitis, bursitis and other soft tissue rheumatism. A non-limiting example of such genetic predisposition is the di-electcons 4 allele of apolipoprotein E, which increases the likelihood of Alzheimer's Disease. Another exemplary genetic predisposition can be a loss of function or null mutation in the lipoprotein lipase gene coding region or promoter, such as, mutations in the coding regions of the lipase gene resulting in the substitutions D9N and N29 1 S. These and other genetic mutations in the lipoprotein lipase gene that increase the risk of cardiovascular diseases, dyslipidemias and dyslipoproteinemias are described in Hayden and Ma, 1992, Mol. Cell Biochem. 113:171 176, herein incorporated by reference in its entirety. Other genetic predispositions include familial combined hyperlipidemia and familial hypercholesterolemia.

In some embodiments, the compounds of the invention or compositions of the invention are provided as a preventative measure to a subject such as a mammal that can having a non-genetic predisposition to cardiometabolic syndrome, conditions or disorders associated with ageing, Alzheimer's Disease, cancer, cardiovascular disease, diabetic nephropathy, diabetic retinopathy, a disorder of glucose metabolism, dyslipidemia, dyslipoproteinemia, reduced bile production, reduced reverse lipid transport, hypertension, impotence, inflammation, insulin resistance, lipid elimination in bile, reduced modulation of C-reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism. Examples of non-genetic predispositions include but are not limited to cardiac bypass surgery and percutaneous transluminal coronary angioplasty, which often lead to restenosis, an accelerated form of atherosclerosis, diabetes in women, which often leads to polycystic ovarian disease, and cardiovascular disease, which often leads to impotence. Accordingly, the compositions described herein can be used for the prevention of one disease or disorder and concurrently treating another (e.g., prevention of polycystic ovarian disease while treating diabetes; prevention of impotence while treating a cardiovascular disease).

In some embodiments, the compositions disclosed herein are provided to a subject to inhibit the onset of insulin resistance in a subject based on criteria including but not limited to family history, diet and drug use. In some embodiments, for example, an individual at risk for developing insulin resistance is identified based on family history, obesity, diabetes, CVD and other associated disease conditions including depression, mental health diseases or disorders, glucose and lipid metabolism disturbances, a diet high in fats, carbohydrates, low dietary fiber, deficiency of essential nutrients, or individuals taking drugs that induces insulin resistance such as a statin drug, a non-steroidal anti-inflammatory drug, a steroid, an oral contraceptive, a hormone replacement therapy drug, a beta blocker, a potassium channel opener, or a diuretic or anti-depressant drugs. Accordingly, some embodiments provide a method for inhibiting the development of drug-induced insulin resistance including administering a dietary chromium complex to an individual receiving a contemporaneous dose of a drug that induces insulin resistance. Advantageously, the amount of chromium complex administered is an amount effective to inhibit the development of insulin resistance.

As used herein, the term “altering lipid metabolism” indicates an observable (measurable) change in at least one aspect of lipid metabolism, including but not limited to total blood lipid content, blood HDL cholesterol, blood LDL cholesterol, blood VLDL cholesterol, blood triglyceride, blood Lp(a), blood apo A-I, blood apo E and blood non-esterified fatty acids, esters of fatty acids, isomers, isoforms and ratios and improving ratios for reducing chronic disease risk but not limited to diabetes, obesity, hypertension, coronary heart disease and cardiovascular disease.

As used herein, the term “altering glucose metabolism” indicates an observable (measurable) change in at least one aspect of glucose metabolism, including but not limited to total blood glucose content, blood insulin, the blood insulin to blood glucose ratio, glycosylated hemoglobin, HOMAIR, beta cell function, composite of insulin sensitivity index, hyperglycemia, hypoglycemia, hormones, enhancing enzyme activities, hormonal balance, lipodystrophy, reducing brain insulin resistance, insulin sensitivity, and oxygen consumption. In some embodiments, the compositions described herein can be used to treat abnormal glucose metabolism that arises due to conditions like polycystic ovary syndrome, HIV, HIV lipodystrophy, Alzheimer's disease, mental health disorders, lipodystrophy, hormonal imbalance conditions, hypertension, obesity and cardiovascular disease and cardiometabolic syndrome.

The present disclosure is based, in part, on the novel and unexpected discovery that when an individual is administered a chromium and histidine, or a chromium histidinate complex alone or concomitantly with another chromium complex, the symptoms and incidence of insulin resistance is lowered. Accordingly, in some embodiments, a method for the inhibition/reduce of insulin resistance and its risk by lowering glucose and lipids and improving insulin sensitivity by including chromium histidinate supplementation is provided. Compositions for the inhibition of insulin resistance in an individual are similarly provided.

As used herein, the term “chromium complexes” or “chromium complex” includes, without limitation, all trivalent chromium complexes, such as chromium picolinate, chromic tripicolinate, chromium nicotinate, chromic polynicotinate, chromium chloride, chromium yeast, and other chromium complexes, whether now known or developed in the future.

“Insulin resistance” refers to a condition characterized by decreased insulin function and hyperinsulinemia, caused or exacerbated by drugs and disease conditions such to obesity, diabetes, CVD in a human or other animal. Examples of drugs which induce insulin resistance include, without limitation, statin drugs such as simvastatin, cerivastatin, pravastatin, atorvastatin, fluvastatin, and lovastatin; non-steroidal anti-inflammatory drugs such as cimicifuga, choline salicylate-magnesium salicylate, diclofenac sodium, diclofenac potassium, diflunisal, etodolac, fenoprofen calcium, floctafenine, flurbiprofen, ibuprofen, indomethacin, ketoprofen, ketorolac tromethamine, magnesium salicylate, mefenamic acid, nabumetone, naproxen, naproxen sodium, oxyphenbutazone, phenylbutazone, piroxicam, salsalate, sodium salicylate, sulindac, tenoxicam, taiprofenic acid, and tolmetin sodium; steroids such as hydrocortisone, dexamethasone, and methylprednisolone; contraceptives including oral contraceptives such as estrogen, progesterone and progestin as well as implantable contraceptives such as levonorgestrel, etonogestrel, nomegestrol acetate, and nestorone; hormone replacement therapy (HRT) drugs including conjugated equine estrogens, esterified estrogens, estradiol, estrone, synthetic conjugated estrogens, estropipate, estropipate, ethinyl estradiol, norethindrone, medroxyprogesterone acetate, progestin, natural progesterone, tamoxifen, testosterone, and raloxifene; beta blocker drugs including acebutolol, atenolol, betaxolol, bucinodol, carteolol, labetalol, metoprolol, nadolol, penbutolol, pindolol, propanolol, and timolol; and diuretics. Three primary types of diuretics exist which include thiazides, loop diuretics, and potassium sparing agents. As used herein, the term “diuretic” or “diuretics” includes, without limitation, hydrochlorothiazide, chlorthalidone, chlorothiazide, indapamide, metolazone, amiloride, spironolactone, triamterene, furosemide, bumetanide, ethacrynic acid, and torsemide. Certain immunosuppressive drugs such as prednisolone, cyclosporin A, and tacromlimus and potassium channel modulators such as nicorandil are also included in the definition of drugs which induce insulin resistance., such as for example antidepressants The above list is provided for example purposes only and it is understood that the definition of “drug which induces insulin resistance” includes those drugs which induce insulin resistance that are not specifically listed above, as well as those drugs which are found to induce insulin resistance, whether in existence today or developed in the future. Examples of diet which induce insulin resistance include diets high in fats, carbohydrates, low dietary fiber, low glycemic index foods, high fructose in the functional foods, beverages, and bars.

The administration of an effective dose of a composition described herein (e.g. chromium histidinate), to subjects who have a diet or take drugs which have been linked with the onset of insulin resistance actually can inhibit or attenuate the onset of insulin resistance. Supplementing the diet or drug therapy with a composition disclosed herein, e.g. a chromium histidinate complex, can inhibit the induction of insulin resistance. By not developing insulin resistance in the first place, the subject avoids exposure to diseases and risks associated with insulin resistance. The subject can also avoid the necessity of taking additional, and sometimes costly, medications to treat the insulin resistance and associated diseases.

Some embodiments provide methods of inhibiting or reducing the risk of insulin resistance through chromium supplementation.

Chromium supplementation includes the administration of chromium histidinate alone or in combination with at least one other chromium complexes to an individual. Advantageously, the chromium complexes are synthetic. The synthesis and use of chromium picolinates, for example, is described in U.S. Pat. Nos. Re 33,988 and 5,087,623, the entire contents of which are hereby incorporated herein by reference in their entirety. Chromic tripicolinate is available from health food stores, drug stores and other commercial sources. The synthesis and use of chromic polynicotinate is described in U.S. Pat. No. 5,194,615.

Inhibition of insulin resistance is accomplished by administering an effective dose of a chromium histidinate complex to an individual as a single composition or in combination with another agent, such as a food, beverage or drug that induces insulin resistance. A subject can begin chromium supplementation at the beginning of their treatment with an agent that induces insulin-resistance. Alternatively, the subject can begin supplementation with a chromium complex after the subject's treatment with an agent that induces insulin resistance (e.g. a food, beverage, drug or the like), but before the subject develops insulin resistance.

Insulin resistance is a key pathogenic parameter of Type 2 diabetes, and clinical interventions that improve insulin sensitivity are considered cornerstones in the management of the disease. In addition, the relationship of insulin resistance to cardiovascular disease and its associated risk factors has been well established over the past few years. Therefore, in some embodiments, methods and compositions for thwarting the development of insulin resistance are provided comprising the administration of a chromium histidinate complex and an agent which inhibits insulin resistance, such as a hypoglycemic drug, e.g. metformin, which inhibits insulin resistance from developing. Combinations of pharmacologic agents (such as sulfonylureas/metformin, sulfonylureas/glitazones, and metformin/glitazones) are highly effective pharmacologic interventions that appear to lower both glucose and insulin levels. Accordingly, some embodiments provide compositions comprising a chromium histidinate complex as described herein in combination the above hyperglycemia and insulin resistance therapies. Some embodiments provide methods of preventing or treating insulin resistance by administering to a subject in need thereof a chromium histidinate complex as described herein in combination the above hyperglycemia and insulin resistance therapies. The skilled artisan will also appreciate that the chromium histidinate complexes described herein can be used in combination with triple drug therapy, such as sulfonylureas/metformin/glitazones, which have been shown to lower clinical glycemia in addition to lowering insulin levels. Hence, in some embodiments, compositions comprising a chromium complex with metformin, sulfonylureas, and glitazones or combinations thereof are administered to a subject taking drugs which induce insulin resistance to inhibit the onset of such insulin resistance.

In some embodiments, provided herein are methods of preventing the development or worsening of conditions associated with the development of insulin resistance or diabetes, such as cardiovascular disease (discussed below), obesity, disease conditions based on ATPIII guidelines due to mental health conditions such as depression, schizophrenia, alzheimers disease and other conditions such HIV and HIV lipodystrophy and polycystic ovary syndrome. The insulin resistance might be due to family history, body weight, diet and drugs.

### Treatment of Cardiovascular Diseases

As discussed above, some embodiments provide methods for the treatment or prevention of a cardiovascular disease, comprising identifying a subject with or at risk of developing cardiovascular disease, and administering to the subject a therapeutically effective amount of a composition comprising, consisting essentially of, or consisting of chromium and histidine, or a chromium histidinate complex and a pharmaceutically acceptable vehicle.

As used herein, the term “cardiovascular diseases” refers to diseases of the heart and circulatory system. Some embodiments provide for the treatment or prevention of arteriosclerosis, atherosclerosis, stroke, ischemia, endothelium dysfunctions, e.g. dysfunctions affecting blood vessel elasticity; peripheral vascular disease, coronary heart disease, myocardial infarction, cerebral infarction, restenosis and the like.

The compositions disclosed herein, e.g. chromium histidinate complexes, are preferably used in methods for treating cardiovascular disease and its related pathologies, including, for example, hypertrophy, hypertension, congestive heart failure, myocardial ischemia, ischemia reperfusion injuries in an organ, arrhythmia, and myocardial infarction. Some embodiments provide methods for treating or preventing cardiovascular disease in a subject by administering to the mammal a therapeutically effective amount of a cardiovascular therapeutic agent and a therapeutically effective amount of a chromium complex disclosed herein. As discussed elsewhere in the specification, the therapeutic agent (e.g., therapeutic cardiovascular agent) can be administered prior to, after, or concurrently, with the chromium complex. Non-limiting examples of therapeutic cardiovascular agents suitable for use in the methods described herein include an angiotensin converting enzyme inhibitor, an angiotensin II receptor antagonist, a calcium channel blocker, an anti-thrombotic agent, a β.-adrenergic receptor antagonist, a vasodilator, a diuretic, an .α-adrenergic receptor antagonist, an antioxidant, or any combination thereof For example, in some embodiments, the therapeutic cardiovascular agent can be PPADS.

### Treatment of Dyslipidemias

Also provided are methods for the treatment or prevention of a dyslipidemia comprising identifying a subject with or at risk of developing dyslipidemia, and administering to the subject a therapeutically effective amount of composition disclosed herein, e.g., a chromium histidinate complex.

As used herein, the term “dyslipidemias” refers to disorders that lead to or are manifested by aberrant levels of circulating lipids. To the extent that levels of lipids in the blood are too high, the compositions of the invention are administered to a patient to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (See, e.g., the world wide web site for the American Heart Organization at americanheart.org/cholesterol/about_level.html and the National Institute of Heath worldwide web site at nhlbi.nih.gov/health/public/heart/chol/hbc_what.html, respectively). At the present time, the recommended level of HDL cholesterol in the blood is above 35 mg/dL; the recommended level of LDL cholesterol in the blood is below 70 mg/dL if they have multiple risk factors; the recommended LDL:HDL cholesterol ratio in the blood is below 5:1, ideally 3.5:1; and the recommended level of free triglycerides in the blood is less than 200 mg/dL.

Dyslipidemias which the compositions of the present invention are useful for preventing or treating include but are not limited to hyperlipidemia and low high density lipoprotein (HDL) cholesterol serum levels. In certain embodiments, the hyperlipidemia for prevention or treatment by the compounds of the present invention is familial hypercholesterolemia; familial combined hyperlipidemia; reduced or deficient lipoprotein lipase levels or activity, including reductions or deficiencies resulting from lipoprotein lipase mutations; hypertriglyceridemia; hypercholesterolemia; high blood levels of urea bodies (e.g. .beta.-OH butyric acid); high blood levels of Lp(a) cholesterol; high blood levels of low density lipoprotein (LDL) cholesterol; high blood levels of very low density lipoprotein (VLDL) cholesterol and high blood levels of non-esterified fatty acids.

Also provided herein are methods for altering lipid metabolism in a subject in need thereof, e.g., reducing LDL in the blood of a subject, reducing free triglycerides in the blood of a subject, increasing the ratio of HDL to LDL in the blood of a subject, and inhibiting saponified and/or non-saponified fatty acid synthesis. Subjects can be identified as needing a reduction in serum LDL levels, an increase in the ratio of serum HDL:LDL cholesterol, or an inhibition of saponified and/or non-saponified fatty acid synthesis using conventional methods known to those skilled in the art. The subjects can be administering to the patient a compound or a composition comprising a compound of the invention in an amount effective alter lipid metabolism.

### Treatment of Dyslipoproteinemias

Also provided herein are methods for the treatment or prevention of a dyslipoproteinemia comprising administering to subject with or at risk of developing dyslipoproteinemia a therapeutically effective amount of a compound or a composition comprising a chromium complex described herein.

As used herein, the term “dyslipoproteinemias” refers to disorders that lead to or are manifested by aberrant levels of circulating lipoproteins. To the extent that levels of lipoproteins in the blood are too high, the compositions described herein can be administered to a subject to restore normal levels. Conversely, to the extent that levels of lipoproteins in the blood are too low, the compositions described herein can be administered to a subject to restore normal levels. Normal levels of lipoproteins are reported in medical treatises known to those of skill in the art.

Accordingly, in some embodiments, provided herein are methods to treat or prevent dyslipoproteinemias including but not limited to high blood levels of LDL, high blood levels of apolipoprotein B (apo B), high blood levels of Lp(a), high blood levels of apo(a), high blood levels of VLDL, low blood levels of HDL, reduced or deficient lipoprotein lipase levels or activity, including reductions or deficiencies resulting from lipoprotein lipase mutations;, hypoalphalipoproteinemia, lipoprotein abnormalities associated with diabetes, lipoprotein abnormalities associated with obesity; lipoprotein abnormalities associated with Alzheimer's Disease, familial combined hyperlipidemia and the like.

Further provided are methods for reducing apo C-II levels in the blood of a subject; reducing apo C-III levels in the blood of a subject; elevating the levels of HDL associated proteins, including but not limited to apo A-I, apo A-II, apo A-IV and apo E in the blood of a subject; elevating the levels of apo E in the blood of a subject, and promoting clearance of triglycerides from the blood of a subject, by identifying a subject in need thereof and administering a compound or a composition comprising a compound described herein in an amount effective to bring about said reduction, elevation or promotion, respectively.

### Treatment of Glucose Metabolism Disorders

Also provided are methods for the treatment or prevention of a glucose metabolism disorder, comprising providing to a subject with or at risk of developing a glucose metabolism disorder a therapeutically effective amount of a compound or a composition comprising an effective amount of a composition described herein, e.g. a chromium complex such as chromium histidinate.

As used herein, the term “glucose metabolism disorders” refers to disorders that lead to or are manifested by aberrant glucose storage and/or utilization. To the extent that indicia of glucose metabolism (i.e., blood insulin, blood glucose) are too high, the compositions of described herein can be administered to a patient to restore normal levels. Conversely, to the extent that indicia of glucose metabolism are too low, the compositions described herein can be administered to a patient to restore normal levels. Normal indicia of glucose metabolism are reported in medical treatises known to those of skill in the art.

Accordingly, provided herein are methods of treating or preventing glucose metabolism disorders such as impaired glucose tolerance, insulin resistance, insulin resistance related breast, colon or prostate cancer, diabetes, including but not limited to type 2 diabetes, type 1 diabetes, gestational diabetes mellitus (GDM), and maturity onset diabetes of the young (MODY), pancreatitis, hypertension, polycystic ovarian disease, HIV lipodystrophy, hormonal imbalance, hypercotisol levers, endothelial dysfunction, Alzheimer's disease, aging and high levels of blood insulin and/or glucose, e.g. hyperglycemia. A subject with a glucose metabolism disorder can be identified, and the subject can be administered a therapeutically effective amount of a composition described herein.

### Treatment of PPAR-Associated Disorders

Also provided are methods for the treatment or prevention of a PPAR-associated disorder, comprising identifying a subject with or at risk of developing a PPAR-associated disorder and administering to the subject a therapeutically effective amount of a composition described herein, e.g. a composition comprising a chromium complex described herein.

As used herein, “treatment or prevention of PPAR associated disorders” encompasses treatment or prevention of rheumatoid arthritis; multiple sclerosis; psoriasis; inflammatory bowel diseases; breast; colon or prostate cancer; low levels of blood HDL; low levels of blood, lymph and/or cerebrospinal fluid apo E; low blood, lymph and/or cerebrospinal fluid levels of apo A-I; high levels of blood VLDL; high levels of blood LDL; high levels of blood triglyceride; high levels of blood apo B; high levels of blood apo C-III and reduced ratio of post-heparin hepatic lipase to lipoprotein lipase activity. HDL can be elevated in lymph and/or cerebral fluid.

### Treatment of Renal Diseases

Further provided are methods for the treatment or prevention of a renal disease, comprising identifying a subject with or at risk of developing a renal disease, and administering to the subject a therapeutically effective amount of a composition described herein, e.g. a comprising a chromium complex such as chromium histidinate.

As used herein, the term “renal diseases” includes but is not limited to glomerular diseases (including but not limited to acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (including but not limited to acute tubular necrosis and acute renal failure, polycystic renal diseasemedullary sponge kidney, medullary cystic disease, nephrogenic diabetes, and renal tubular acidosis), tubulointerstitial diseases (including but not limited to pyelonephritis, drug and toxin induced tubulointerstitial nephritis, hypercalcemic nephropathy, and hypokalemic nephropathy) acute and rapidly progressive renal failure, chronic renal failure, nephrolithiasis, or tumors (including but not limited to renal cell carcinoma and nephroblastoma). In a most preferred embodiment, renal diseases that are treated by the compounds of the present invention are vascular diseases, including but not limited to hypertension, nephrosclerosis, microangiopathic hemolytic anemia, atheroembolic renal disease, diffuse cortical necrosis, and renal infarcts.

### Treatment of Cancer

Provided herein are methods for the treatment or prevention of cancer, comprising identifying a subject with or at risk of developing cancer and administering to the subject a therapeutically effective amount of a composition described herein, e.g., a composition comprising a chromium complex described herein.

As used herein, the term “treatment or prevention of cancer” can refer to the teratment or prevention of, for example, solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas cystadenocarcinoma, medullary carcinoma, bronchogenic carcinoma, renal cell carcinoma, hepatoma bile duct carcinoma choriocarcinoma seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme astrocytoma medulloblastoma, craniopharyngioma, ependymoma, pinealoma, hemangioblastoma acoustic neuroma, oligodendroglioma, meningioma, skin cancer, melanoma, neuroblastoma, retinoblastoma, Blood-borne cancers, including but not limited to: acute lymphoblastic B-cell leukemia, acute lymphoblastic T-cell leukemia, acute myeloblastic leukemia, “AML,” acute promyelocytic leukemia “APL,” acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia, “CML,” chronic lymphocytic leukemia, “CLL,” hairy cell leukemia, multiple myeloma Acute and chronic leukemias, Lymphoblastic myelogenous leukemias, lymphocytic myelocytic leukemias, Lymphomas: such as Hodgkin's disease, non-Hodgkin's Lymphoma, Multiple myeloma, Waldenstrom's macroglobulinemia, Heavy chain disease, and Polycythemia vera.

Cancer, including, but not limited to, a tumor, metastasis, or any disease or disorder characterized by uncontrolled cell growth, can be treated or prevented by administration of a composition disclosed herein, e.g., a composition comprising a chromium complex such as chromium histidinate.

### Treatment of Other Diseases

Also provided herein are methods for the treatment or prevention of other diseases or disorders including Alzheimer's Disease, Syndrome X, septicemia, thrombotic disorders, obesity, pancreatitis, hypertension, inflammation, and impotence, comprising administering to a patient a therapeutically effective amount of a composition comprising, consisting essentially of, or consisting of a chromium complex such as chromium histidinate.

As used herein, “treatment or prevention of Alzheimer's Disease” encompasses treatment or prevention of lipoprotein abnormalities associated with Alzheimer's Disease.

As used herein, “treatment or prevention of Syndrome X or Metabolic Syndrome” encompasses treatment or prevention of a symptom thereof, including but not limited to impaired glucose tolerance, hypertension and dyslipidemia/dyslipoproteinemia.

As used herein, “treatment or prevention of septicemia” encompasses treatment or prevention of septic shock.

As used herein, “treatment or prevention of thrombotic disorders” encompasses treatment or prevention of high blood levels of fibrinogen and promotion of fibrinolysis.

In addition to treating or preventing obesity, the compositions of the invention can be administered to an individual to promote weight reduction of the individual.

As used herein, “treatment or prevention of diabetic nephropathy” encompasses treating or preventing kidney disease that develops as a result of diabetes mellitus (DM). Diabetes mellitus is a disorder in which the body is unable to metabolize carbohydrates (e.g., food starches, sugars, cellulose) properly. The disease is characterized by excessive amounts of sugar in the blood (hyperglycemia) and urine; inadequate production and/or utilization of insulin; and by thirst, hunger, and loss of weight. Thus, the compositions disclosed herein can also be used to treat or prevent diabetes mellitus.

As used herein, “treatment or prevention of diabetic retinopathy” encompasses treating or preventing complications of diabetes that lead to or cause blindness. Diabetic retinopathy occurs when diabetes damages the tiny blood vessels inside the retina, the light-sensitive tissue at the back of the eye.

As used herein, “treatment or prevention of impotence” includes treating or preventing erectile dysfunction, which encompasses the repeated inability to get or keep an erection firm enough for sexual intercourse. The word “impotence” can also be used to describe other problems that interfere with sexual intercourse and reproduction, such as lack of sexual desire and problems with ejaculation or orgasm. The term “treatment or prevention of impotence includes, but is not limited to impotence that results as a result of damage to nerves, arteries, smooth muscles, and fibrous tissues, or as a result of disease, such as, but not limited to, diabetes, kidney disease, chronic alcoholism, multiple sclerosis, atherosclerosis, vascular disease, and neurologic disease.

As used herein, “treatment or prevention of hypertension” encompasses treating or preventing blood flow through the vessels at a greater than normal force, which strains the heart; harms the arteries; and increases the risk of heart attack, stroke, and kidney problems. The term hypertension includes, but is not limited to, cardiovascular disease, essential hypertension, hyperpiesia, hyperpiesis, malignant hypertension, secondary hypertension, or white-coat hypertension.

As used herein, “treatment or prevention of inflammation” encompasses treating or preventing inflammation diseases including, but not limited to, chronic inflammatory disorders of the joints including arthritis, e.g., rheumatoid arthritis and osteoarthritis; respiratory distress syndrome, inflammatory bowel diseases such as ileitis, ulcerative colitis and Crohn's disease; and inflammatory lung disorders such as asthma and chronic obstructive airway disease, inflammatory disorders of the eye such as corneal dystrophy, trachoma, onchocerciasis, uveitis, sympathetic ophthalmitis, and endophthalmitis; inflammatory disorders of the gum, e.g., periodontitis and gingivitis; tuberculosis; leprosy; inflammatory diseases of the kidney including glomerulonephritis and nephrosis; inflammatory disorders of the skin including acne, sclerodermatitis, psoriasis, eczema, photoaging and wrinkles; inflammatory diseases of the central nervous system, including AIDS-related neurodegeneration, stroke, neurotrauma, Alzheimer's disease, encephalomyelitis and viral or autoimmune encephalitis; autoimmune diseases including immune-complex vasculitis, systemic lupus and erythematodes; systemic lupus erythematosus (SLE); and inflammatory diseases of the heart such as cardiomyopathy.

### Combination Therapy

In certain embodiments, the compounds and compositions disclosed herein can be used in combination therapy with at least one other therapeutic agent. The compound of the invention and the therapeutic agent can act additively or, more preferably, synergistically. In a preferred embodiment, a compound or a composition comprising a compound of the invention is administered concurrently with the administration of another therapeutic agent, which can be part of the same composition as the compound of the invention or a different composition. In another embodiment, a compound or a composition comprising a compound of the invention is administered prior or subsequent to administration of another therapeutic agent. As many of the disorders for which the compounds and compositions disclosed herein are useful in treating are chronic disorders, in one embodiment combination therapy involves alternating between administering a compound or a composition comprising a chromium complex described herein, such as chromium histidinate, and a composition comprising another therapeutic agent, e.g., to minimize the toxicity associated with a particular drug. The duration of administration of each composition, drug or therapeutic agent can be, e.g., one month, three months, six months, or a year. In certain embodiments, when a composition described herein is administered concurrently with another therapeutic agent that potentially produces adverse side effects including but not limited to toxicity, the therapeutic agent can advantageously be administered at a dose that falls below the threshold at which the adverse side is elicited. The standard dosage for the therapeutic agents discussed below are known to those skilled in the art.

The present compositions can be administered together with a statin. Statins for use in combination with the compounds and compositions of the invention include but are not limited to atorvastatin, pravastatin, fluvastatin, lovastatin, simvastatin, and cerivastatin.

The present compositions can also be administered together with a PPAR agonist, for example a thiazolidinedione or a fibrate. Thiazolidinediones for use in combination with the compounds and compositions of the invention include but are not limited to 5 ((4 (2 (methyl 2 pyridinylamino)ethoxy)phenyl)methyl) 2,4 thiazolidinedione, troglitazone, pioglitazone, ciglitazone, WAY 120,744, englitazone, AD 5075, darglitazone, and rosiglitazone. Fibrates for use in combination with the compounds and compositions of the invention include but are not limited to gemfibrozil, fenofibrate, clofibrate, or ciprofibrate. As mentioned previously, a therapeutically effective amount of a fibrate or thiazolidinedione often has toxic side effects. Accordingly, in a preferred embodiment of the present invention, when a composition described herein is administered in combination with a PPAR agonist, the dosage of the PPAR agonist is below that which is accompanied by toxic side effects.

The present compositions can also be administered together with a bile acid binding resin. Bile acid binding resins for use in combination with the compounds and compositions of the invention include but are not limited to cholestyramine and colestipol hydrochloride. The present compositions can also be administered together with niacin or nicotinic acid. The present compositions can also be administered together with a RXR agonist. RXR agonists for use in combination with the compounds of the invention include but are not limited to LG 100268, LGD 1069, 9-cis retinoic acid, 2 (1 (3,5,5,8,8 pentamethyl 5,6,7,8 tetrahydro 2 naphthyl) cyclopropyl) pyridine 5 carboxylic acid, or 4 ((3,5,5,8,8 pentamethyl 5,6,7,8 tetrahydro 2 naphthyl)2 carbonyl) benzoic acid. The present compositions can also be administered together with an anti-obesity drug. Anti-obesity drugs for use in combination with the compositions and compounds described herein (e.g. compositions comprising chromium complexes such as chromium histidinate) include but are not limited to .beta.-adrenergic receptor agonists, preferably beta.-3 receptor agonists, fenfluramine, dexfenfluramine, sibutramine, bupropion, fluoxetine, and phentermine. The compositions disclosed herein can also be administered together with a hormone. Hormones for use in combination with the compounds of the invention include but are not limited to thyroid hormone, estrogen and insulin. Non-limiting examples of insulins include injectable insulin, transdermal insulin, inhaled insulin, or any combination thereof As an alternative to insulin, an insulin derivative, secretagogue, sensitizer or mimetic can be used. Insulin secretagogues for use in combination with the compounds of the invention include but are not limited to forskolin, dibutryl cAMP or isobutylmethylxanthine (IBMX).

The present compositions can also be administered together with a phosphodiesterase type 5 (“PDE5”) inhibitor to treat or prevent disorders, such as but not limited to, impotence. In a particular, embodiment the combination is a synergistic combination of a composition of the invention and a PDE5 inhibitor.

The present compositions can also be administered together with a tyrophostine or an analog thereof Tyrophostines for use in combination with the compounds of the invention include but are not limited to tryophostine 51.

The present compositions can also be administered together with sulfonylurea-based drugs. Sulfonylurea-based drugs for use in combination with the compounds of the invention include, but are not limited to, glisoxepid, glyburide, acetohexamide, chlorpropamide, glibornuride, tolbutamide, tolazamide, glipizide, gliclazide, gliquidone, glyhexamide, phenbutamide, and tolcyclamide. The present compositions can also be administered together with a biguanide. Biguanides for use in combination with the compounds of the invention include but are not limited to metformin, phenformin and buformin.

The present compositions can also be administered together with an α-glucosidase inhibitor. α-glucosidase inhibitors such as, for example acarbose, miglitol and the like.

The present compositions can also be administered together with an apo A-I agonist. For example, in some embodiments, the compositions described herein (e.g. compositions comprising chromium complexes such as chromium histidinate) can be administered with the Milano form of apo A-I (apo A-IM). The apo A-IM can be produced by the method of U.S. Pat. No. 5,721,114 to Abrahamsen, the entire disclosure of which is herein expressly incorporated by reference in its entirety. In some embodiments, the apo A-I agonist can be a peptide agonist. Apo A-I peptide agonists can be peptides disclosed in U.S. Pat. No. 6,004,925 or 6,037,323 to Dasseux, the entire disclosures of which are herein expressly incorporated by reference in their entireties.

The present compositions can also be administered together with apolipoprotein E (apo E).

In yet other embodiments, the present compositions can be administered together with an HDL-raising drug; an HDL enhancer; or a regulator of the apolipoprotein A-I, apolipoprotein A-IV and/or apolipoprotein genes.

In one embodiment, the other therapeutic agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron.

In some embodiments, the other therapeutic agent can be an hematopoietic colony stimulating factor. For example, some embodiments provide for the administration of a composition described herein (e.g. a chromium complex such as chromium histidinate) and a hematopoietic colony stimulating factors such as filgrastim, sargramostim, molgramostim, erythropoietin α or the like.

In some embodiments, the compositions described herein can be administered with another therapeutic agent such as an opioid or non-opioid analgesic agent. Suitable opioid analgesic agents include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

### Combination Therapy of Cardiovascular Diseases

As discussed above, the compositions described herein (e.g., compositions comprising a chromium complex such as chromium histidinate) can be administered together with a known cardiovascular therapeutics. Exemplary cardiovascular drugs for use in combination with the compounds described herein include but are not limited to peripheral antiadrenergic drugs, centrally acting antihypertensive drugs (e.g., methyldopa, methyldopa HCl), antihypertensive direct vasodilators (e.g., diazoxide, hydralazine HCl), drugs affecting renin-angiotensin system, peripheral vasodilators, phentolamine, antianginal drugs, cardiac glycosides, inodilators (e.g., aminone, milrinone, enoximone, fenoximone, imazodan, sulmazole), antidysrhythmic drugs, calcium entry blockers, ranitine, bosentan, and rezulin.

### Surgical Uses

Cardiovascular diseases such as atherosclerosis often require surgical procedures such as angioplasty. Angioplasty is often accompanied by the placement of a reinforcing a metallic tube shaped structure known as a “stent” into a damaged coronary artery. For more serious conditions, open heart surgery such as coronary bypass surgery can be required. These surgical procedures entail using invasive surgical devices and/or implants, and are associated with a high risk of restenosis and thrombosis. Accordingly, the compounds and compositions of the invention can be used as coatings on surgical devices (e.g., catheters) and implants (e.g., stents) to reduce the risk of restenosis and thrombosis associated with invasive procedures used in the treatment of cardiovascular diseases.

### Veterinary and Livestock Uses

Compositions described herein can be administered to an animal or non-human animal for a veterinary use for treating or preventing a disease or disorder disclosed herein.

In some embodiments, the non-human animal is a household pet. In some embodiments embodiment, the non-human animal is a livestock animal. In some embodiments, the non-human animal is a mammal, such as a cow, horse, sheep, pig, cat, dog, mouse, rat, rabbit, or guinea pig. In some embodiments, the non-human animal is a fowl species, most preferably a chicken, turkey, duck, goose, or quail.

In addition to veterinary uses, the compositions disclosed herein can be used to reduce the fat content of livestock to produce leaner meats. Alternatively, the compositions disclosed herein can be used to reduce the cholesterol content of eggs by administering the compounds to a chicken, quail, or duck hen. For non-human animal uses, the compositions disclosed herein can be administered via the animals' feed or orally as a drench composition.

### Therapeutic/Prophylactic Administration and Compositions

As discussed herein, the compositions disclosed herein (e.g. compositions comprising, consisting essentially of, or consisting of a chromium complex such as chromium histidinate) are useful in veterinary and human medicine. As described above, the compounds and compositions described herein are useful for the treatment or prevention of cardiometabolic syndrome, aging, Alzheimer's Disease, cancer, cardiovascular disease, diabetic nephropathy, diabetic retinopathy, a disorder of glucose metabolism, dyslipidemia, dyslipoproteinemia, hypertension, impotence, inflammation, insulin resistance, lipid elimination in bile, modulating C reactive protein, obesity, oxysterol elimination in bile, pancreatitis, Parkinson's disease, a peroxisome proliferator activated receptor-associated disorder, phospholipid elimination in bile, renal disease, septicemia, metabolic syndrome disorders (e.g., Syndrome X), a thrombotic disorder, enhancing bile production,-enhancing reverse lipid transport, inflammatory processes and diseases like gastrointestinal disease, irritable bowel syndrome (IBS), inflammatory bowel disease (e.g., Crohn's Disease, ulcerative colitis), arthritis (e.g., rheumatoid arthritis, osteoarthritis), autoimmune disease (e.g., systemic lupus erythematosus), scleroderma, ankylosing spondylitis, gout and pseudogout, muscle pain: polymyositis/polymyalgia rheumatica/fibrositis; infection and arthritis, juvenile rheumatoid arthritis, tendonitis, bursitis and other soft tissue rheumatism.

Provided herein are methods of treatment and prophylaxis of the conditions enumerated above by providing to a subject of a therapeutically effective amount of a composition disclosed herein. The mammal can be an animal, such as t a cow, horse, sheep, pig, chicken, turkey, quail, cat, dog, mouse, rat, rabbit, guinea pig, etc., or a human human.

The compositions disclosed herein are useful for methods for treating diabetes and its related pathologies, cardiovascular and related diseases, such as, for example, diabetes retinopathy, diabetes nephropathy, diabetes neuropathy, diabetes foot problems, diabetes infections and inflammations, diabetes with cardiovascular complications such as hypertrophy, hypertension, congestive heart failure, myocardial ischemia, ischemia reperfusion injuries in an organ, arrhythmia, and myocardial infarction. Some embodiments provide methods of treating cardiovascular disease in a mammal by concurrently administering to the mammal a therapeutically effective amount of a combination of a compound suitable for use in methods of the invention and a therapeutic cardiovascular compound such as chromium histidine or chromium complex. Therapeutic cardiovascular compounds suitable for use in methods of the invention include an angiotensin converting enzyme inhibitor, an angiotensin II receptor antagonist, a calcium channel blocker, an anti-thrombotic agent, a β.-adrenergic receptor antagonist, a vasodilator, a diuretic, an α.-adrenergic receptor antagonist, an antioxidant, and a mixture thereof In some embodiments, the chromium histidinate compounds and compositions of the disclosed herein are administered with therapeutic diabetes reducing agents.

The compounds disclosed herein are useful for the methods for treating obesity and related pathologies, obesity related to complications such as diabetes, diabetes risk factors, leptin resistance, abdominal fat distribution, cardiovascular disease and its related pathologies, cardiovascular and related diseases, such as, for example, hypertrophy, hypertension, congestive heart failure, myocardial ischemia, ischemia reperfusion injuries in an organ, arrhythmia, and myocardial infarction. One embodiment is directed to a method of treating obesity and its associated complications such as diabetes, cardiovascular disease and insulin resistance in a mammal by concurrently administering to the mammal a therapeutically effective amount of a combination of a compound suitable for use in methods of the invention and a therapeutic cardiovascular compound such as chromium histidine or chromium complex. Therapeutic chromium histidine and in combination with suitable drug for use in methods of the invention include an angiotensin converting enzyme inhibitor, an angiotensin II receptor antagonist, a calcium channel blocker, an anti-thrombotic agent, a β.-adrenergic receptor antagonist, a vasodilator, a diuretic, an α.-adrenergic receptor antagonist, an antioxidant, antihyperglycemic drugs, insulin, antiobesity drugs, antidepressants etc. and a mixture thereof In some embodiments, the therapeutic doses of drugs alone or in combination with chromium complex

Other methods will be known to the skilled artisan and are within the scope of the invention

The following examples are provided by way of illustration and not limitation.

### Example 1

### Effects of Chromium Histidinate on Media Glucose Concentration and Triglyceride Secretion In Vitro

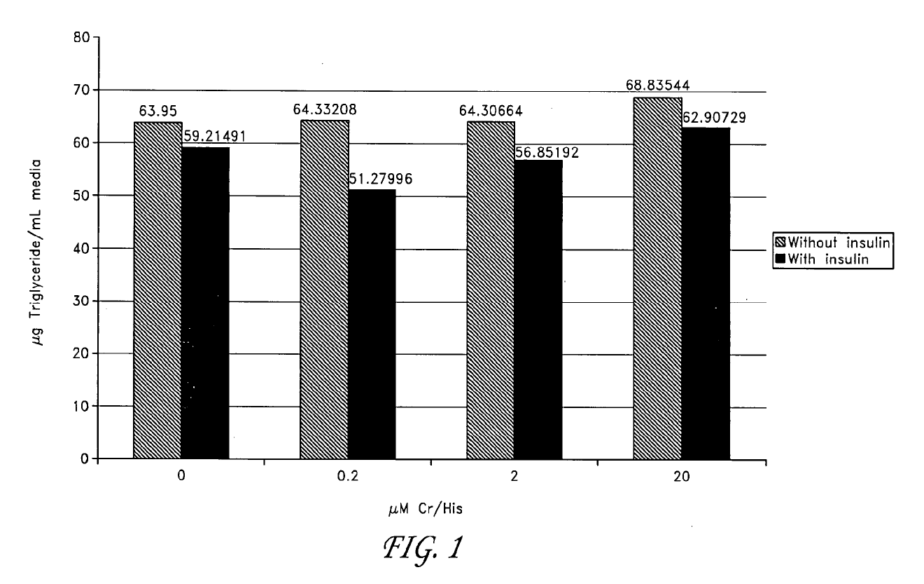

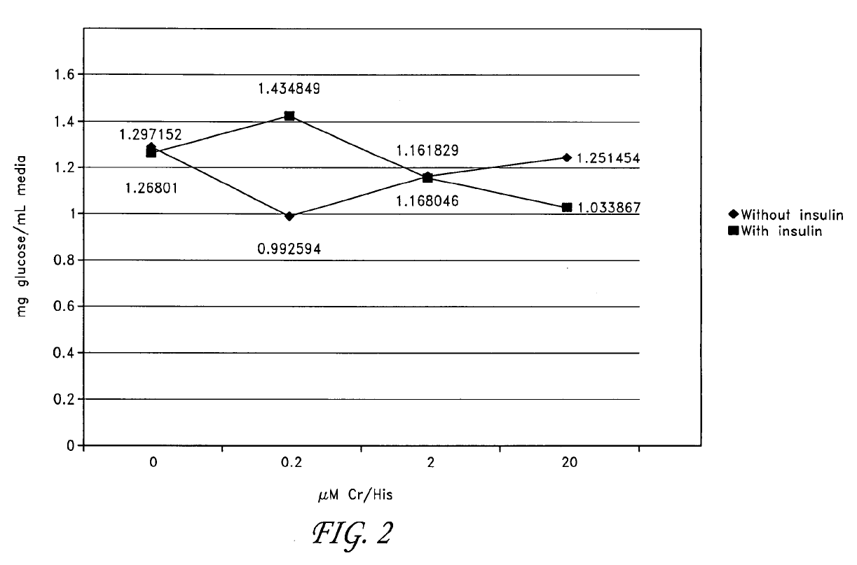

Hep G2 cells are liver cells derived from a human hepatoblastoma that is free of known hepatotropic viral agents. This cell line expresses a wide variety of liver-specific metabolic functions and is used as a model system to study cholesterol and triglyceride metabolism. The effects of chromium histidinate on triglyceride secretion and on media glucose levels, the HepG2 cell line was grown in culture media with or without insulin in the presence of 0, 0.2, 2, or 20 μM chromium histidinate. Media glucose levels and triglyceride levels were measured using standard protocols. Specifically, triglyceride levels were measured spectrophotometrically through hydrolysis by lipase and coupled enzyme reactions on the resulting glycerol. The results of the triglyceride assay are shown in FIG. 1. Glucose levels were measured spectrophotometrically using the glucose oxidase method, with a standard dilution curve serving to calibrate the measurements. The results of the glucose assay are shown in FIG. 2.

In the presence of insulin, chromium histidinate at the lowest dose (0.2 μM) significantly decreased triacylglycerol. Also, at this dose of chromium histidinate in the absence of insulin, there was a significant decrease in glucose in the media. The differences were statistically significant (p<0.05) when compared to the control groups.

### Example 2

### Effects of Chromium Histidinate on Glucose and Lipid Metabolism In Vivo

The following example describes experiments showing the effects of chromium histidinate supplementation on the glucose and lipid metabolism in rat model systems for insulin resistance and diabetes. The studies also assessed the effects of chromium histidine supplementation on histopathological status of tissues in STZ diabetic rats.

### Animals

Wistar rats were reared at the temperature of (22±2° C.), humidity (55±5%) and a 12/12 h light/dark cycle. Pellet food and water were provided ad libitum.

### Induction of Type II Diabetes

Fat-fed/STZ treated rats provide an animal model for type 2 diabetes that simulates the human syndrome, and is suitable for the testing of antidiabetic compounds (See, e.g. Reed et al. (2000) Metabolism 49(11):1390-1394). Rats fed a high fat diet can be used as a model system for insulin resistance. Ten Wistar rats (55 days old) in each group were treated as follows:

Group 1: Control rats were fed standard diet (12% of calories as fat) for 12 weeks.

Group 2: Control rats were fed standard diet+chromium histidinate for 12 weeks.

Group 3: Rats were fed high fat diet (40% of calories as fat) for 12 weeks.

Group 4: Rats were fed high-fat diet (40% of calories as fat) and chromium histidinate (approx. 110 mcg/kg body·d) was included into water for 12 weeks.

Group 5: Rats were fed high-fat diet (40% of calories as fat) for 2 weeks and then injected with streptozotocin (STZ, 40 mg/kg i.p.) for 12 weeks.

Group 6: Rats were fed high-fat diet (40% of calories as fat) for 2 weeks and then injected with streptozotocin (STZ, 40 mg/kg i.p.) and chromium histidinate was included into water at a concentration of 110-mcg/kg body.d for 10 weeks.

Before STZ injection glucose concentrations of rats were measured and compared to controls. After the injection of STZ, animals exhibiting fasting glucose levels >140 mg/dl was considered as neonatal-STZ (nSTZ)-diabetic resembling type II diabetes in humans, plasma insulin concentrations in response to oral glucose (2 g/kg) was evaluated.

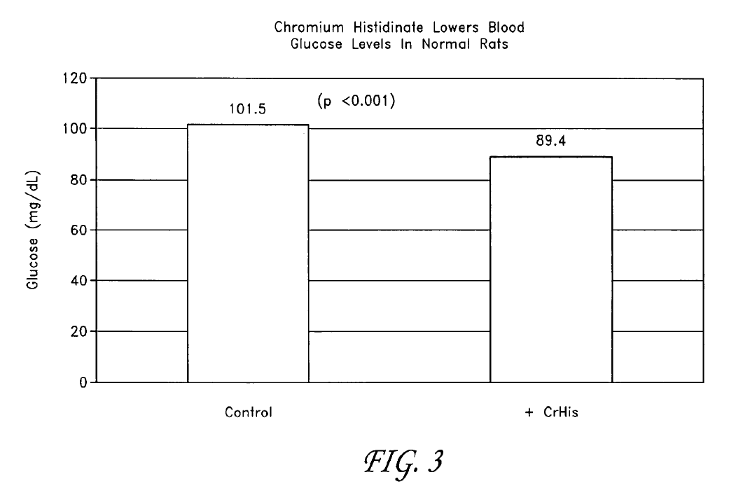

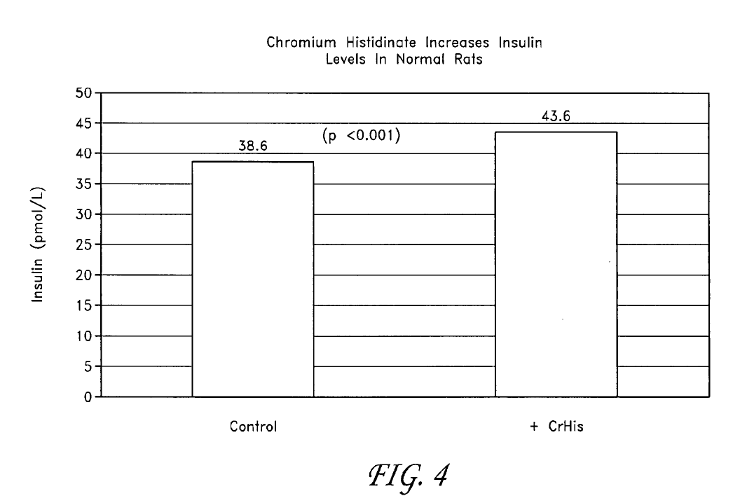

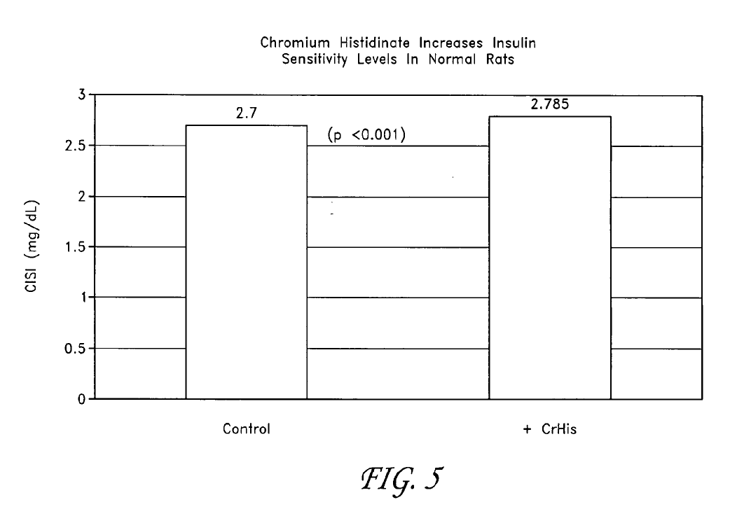

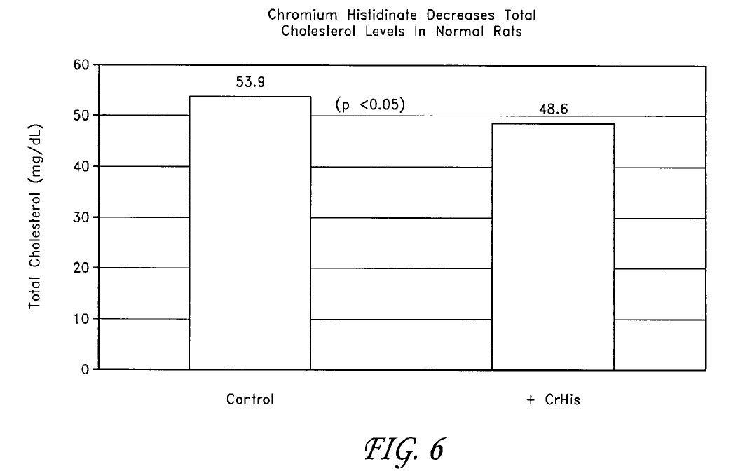

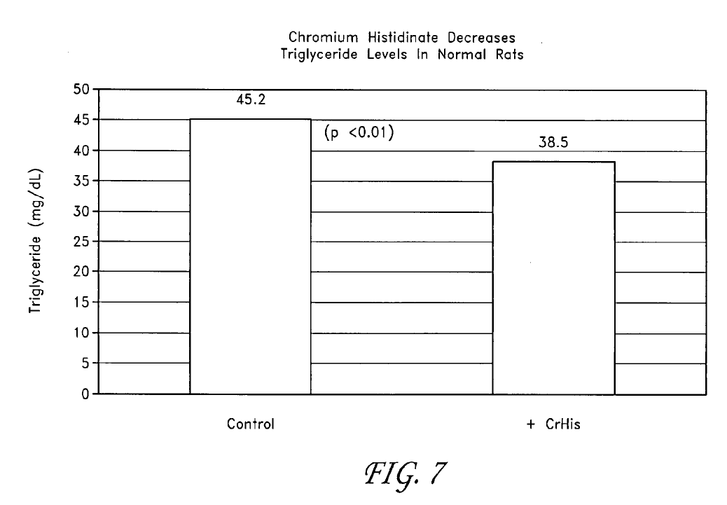

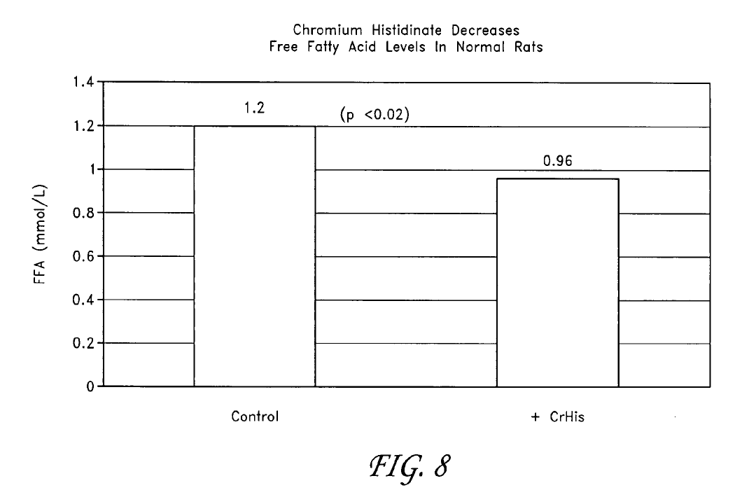

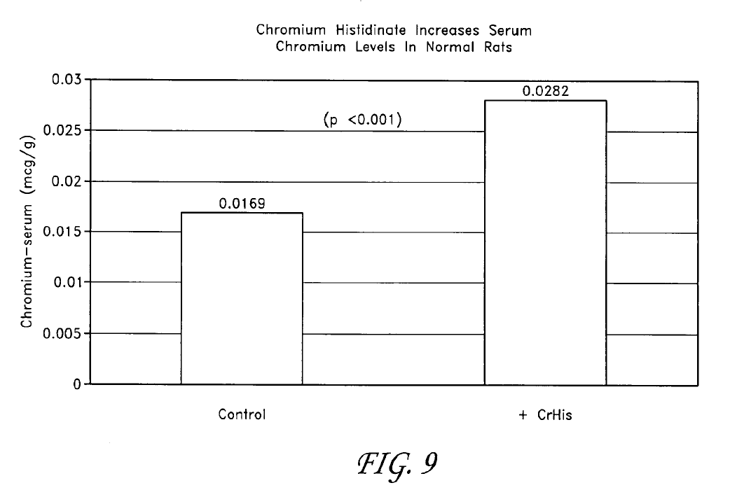

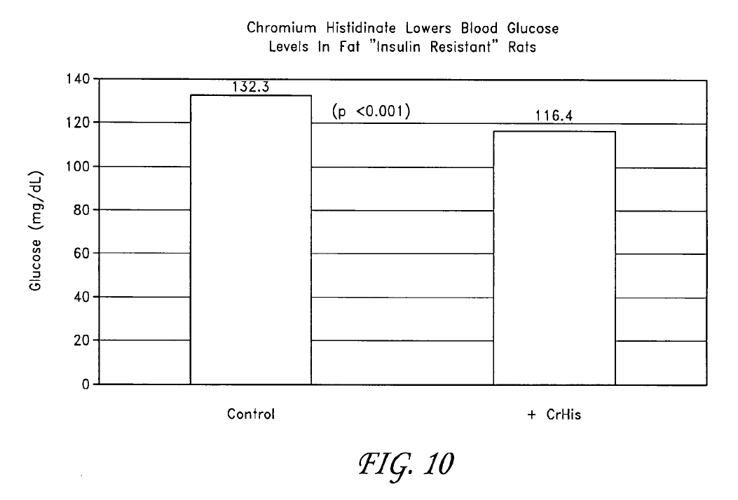

The results of study groups 1 and 2 are presented in FIGS. 3-10. These data show that chromium histidinate lowers serum glucose levels, increases insulin levels, increases insulin sensitivity, decreases total serum cholesterol levels, decreases serum triglyceride levels, decreases free fatty acid levels, and increases serum chromium levels in normal rats.

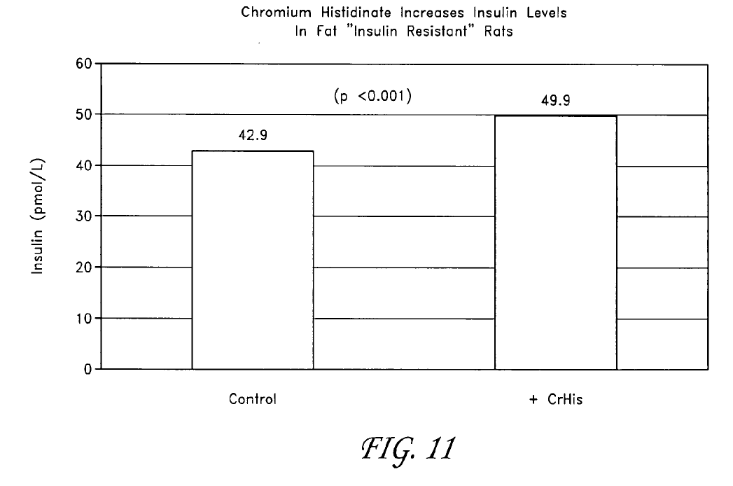

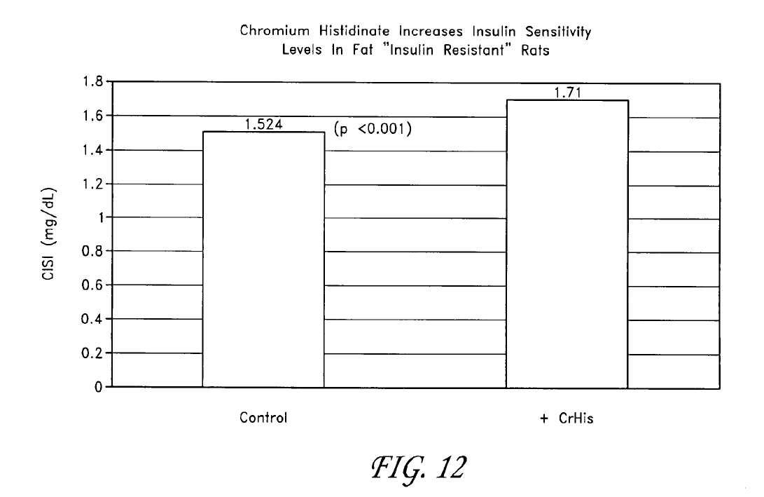

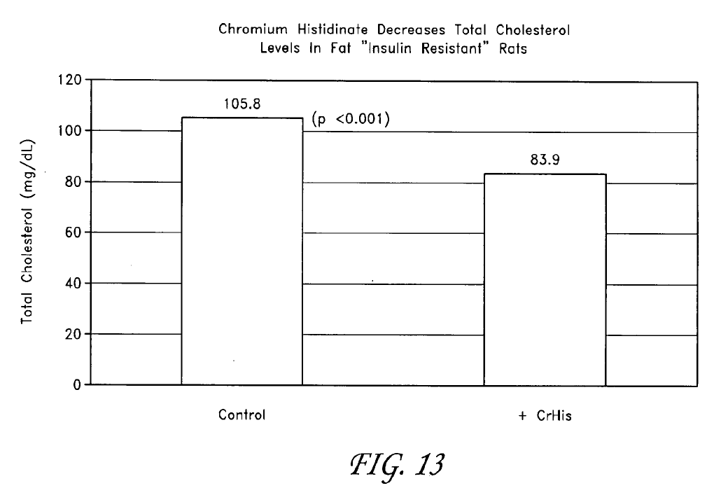

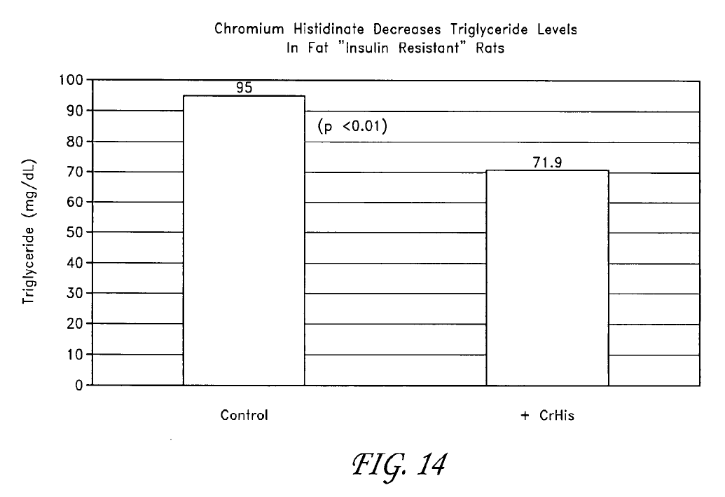

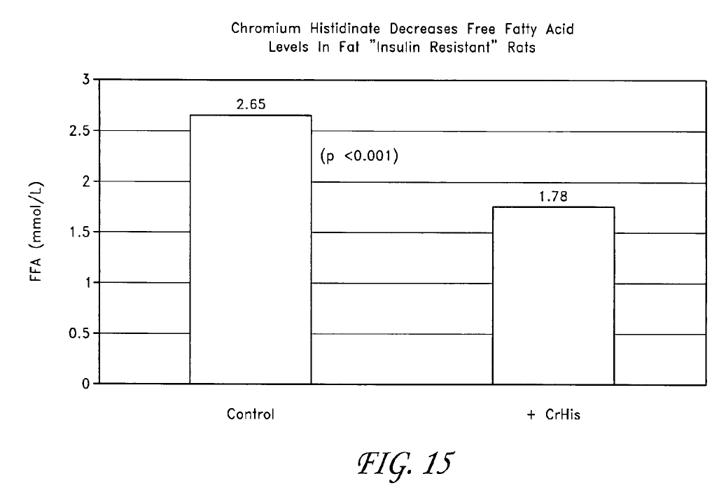

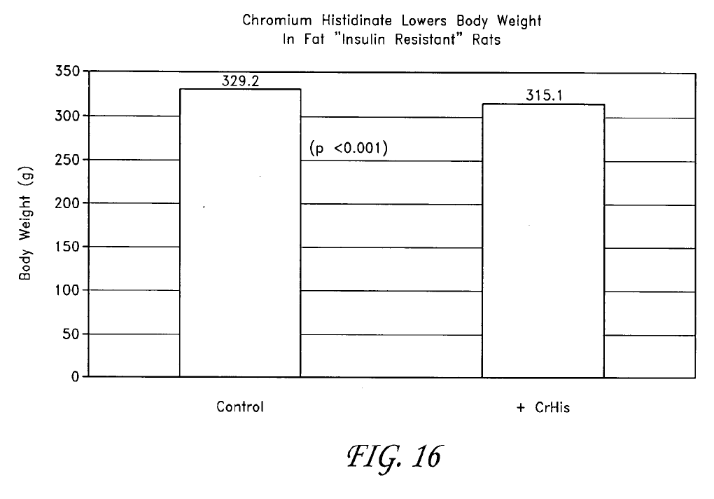

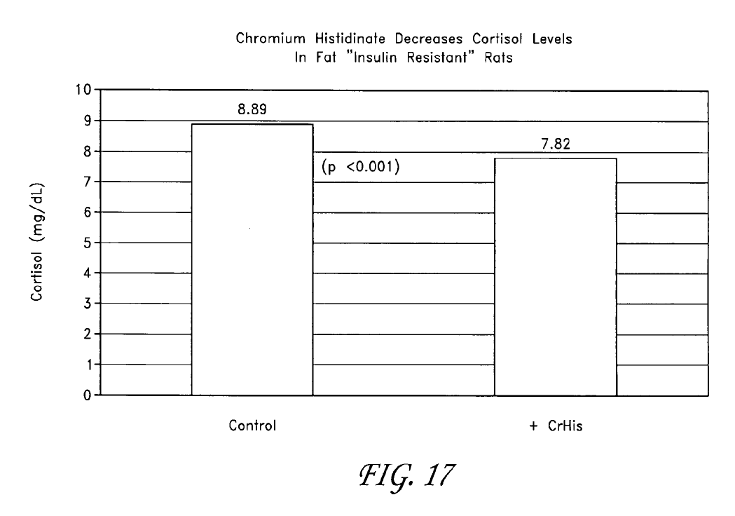

The results of study groups 3 and 4 are presented in FIGS. 11-17. These data show that chromium histidinate lowers serum glucose levels, increases insulin levels, increases insulin sensitivity, decreases total serum cholesterol levels, decreases triglyceride levels, decreases free fatty acid levels, significantly lowers body weight, and decreases cortisol levels in insulin resistant rats.

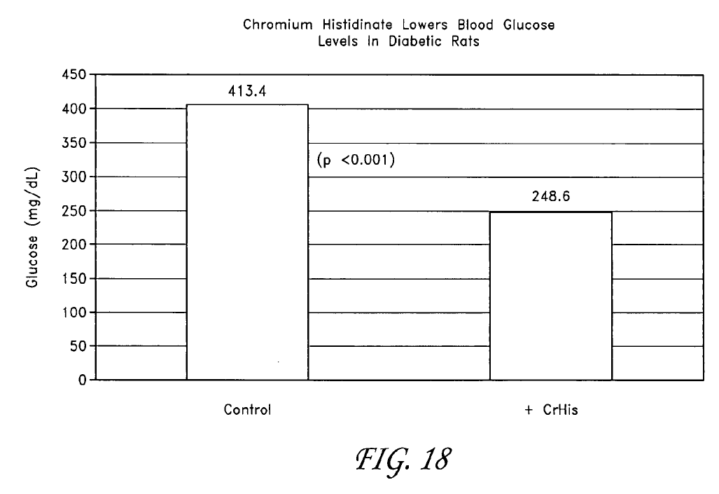

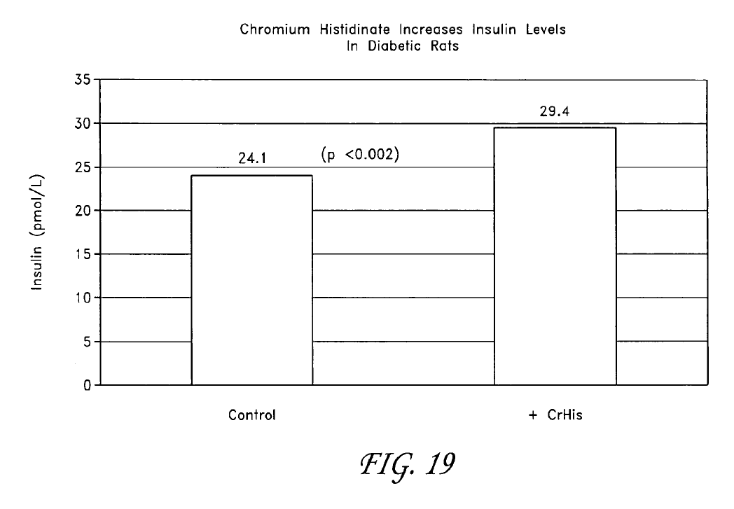

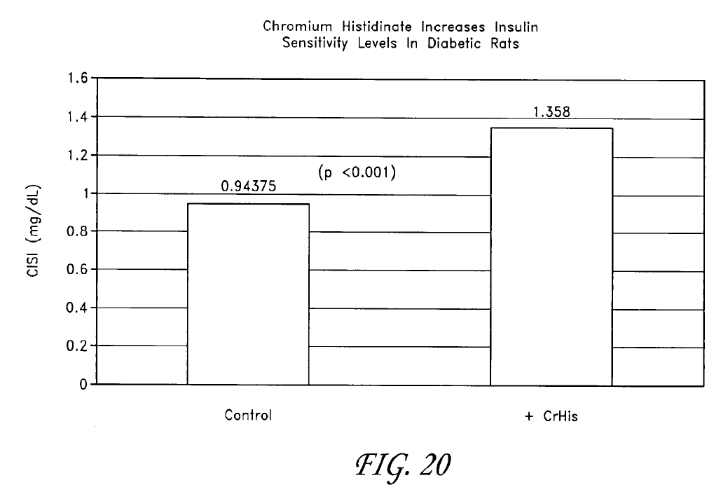

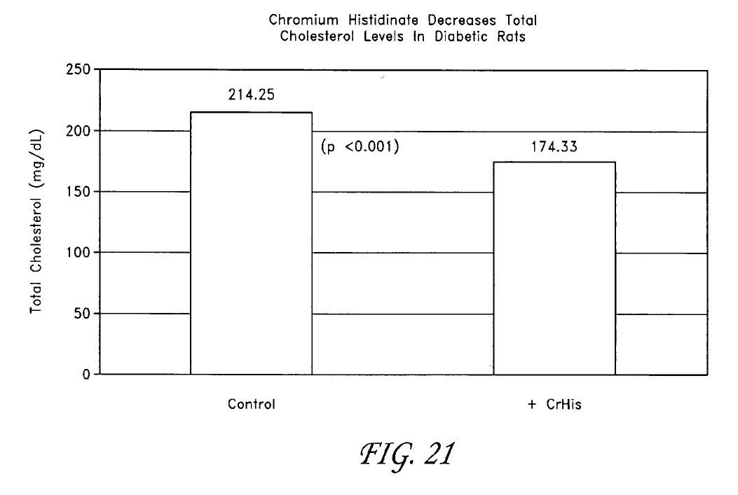

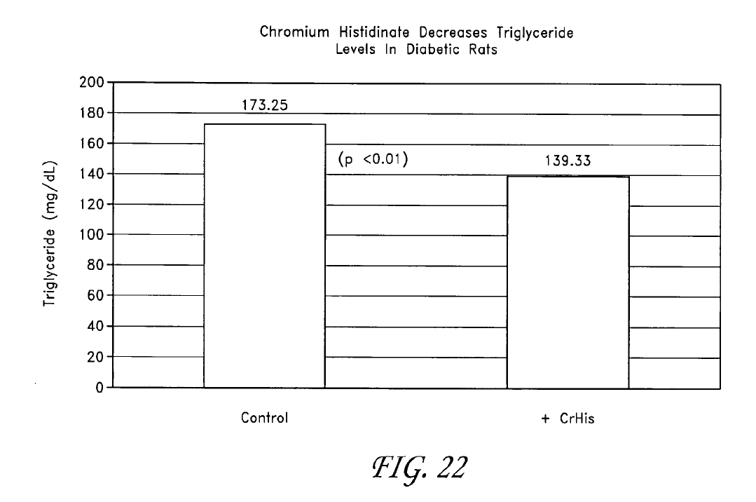

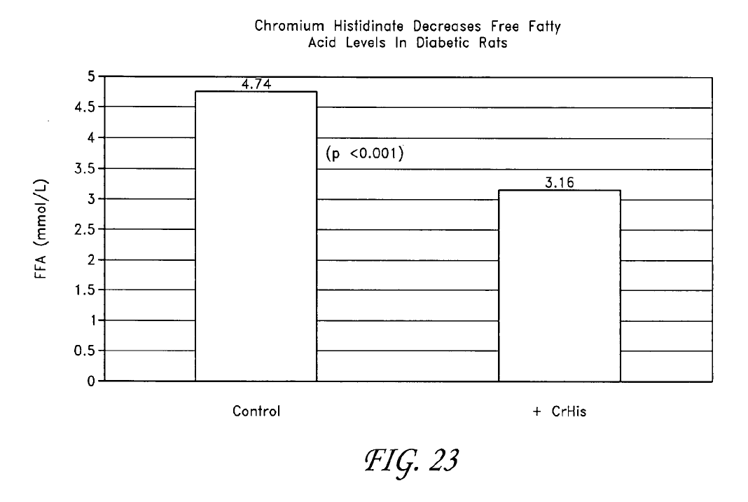

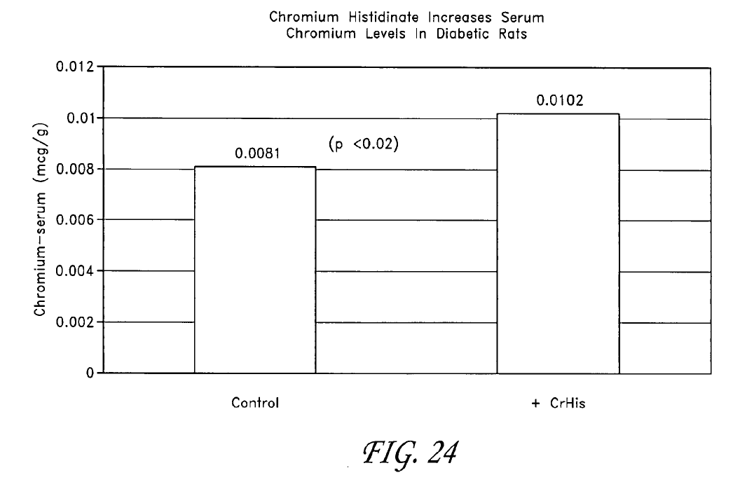

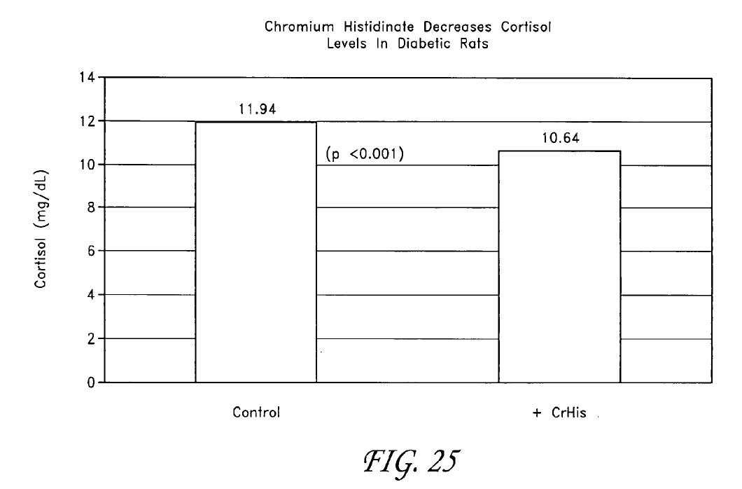

The results of study groups 5 and 6 are presented in FIGS. 18-25. These data show that chromium histidinate lowers serum glucose levels, increases insulin levels, increases insulin sensitivity, decreases total serum cholesterol levels, decreases triglyceride levels, decreases free fatty acid levels, significantly lowers body weight, and decreases cortisol levels in diabetic rats.

### Example 4

### Treatment of Cardiometabolic Syndrome with Chromium Histidinate

A subject is identified as having cardiometabolic syndrome. The subject presents with one or more symptoms associated with cardiometabolic syndrome such as obesity, hypertension, dyslipidemia, impaired glucose tolerance, diabetes, an increase in C-reactive protein, and increase in TNFα, an increase in IL-6, an increase in IL-10, or an increase in oxidative stress.

The individual is administered between 50 μg and 5000 μg chromium histidinate complex/day, orally. The chromium histidinate is administered orally. After a period of time, a reduction in one or more of the symptoms is observed.

### Example 5

### Prevention of Insulin Resistance Associated with Drug Therapy

A subject is identified that is taking a drug therapy associated with the development of insulin resistance. The subject can be presently taking a statin drug, a non-steroidal anti-inflammatory drug, a contraceptive (e.g. an oral contraceptive), hormone replacement therapy, beta blocker, thiazides, diuretics, antidepressants, or any combination thereof

The subject is administered an effective amount of chromium and histidine e.g. to provide between about 50 μg and 5000 μg chromium) concomitantly with the insulin-resistance inducing drug therapy. The chromium and histidine is administered substantially at the same time as the drug therapy that induces insulin resistance. The subject does not develop signs of insulin resistance, or exhibits a lesser degree of insulin resistance compared to individuals not receiving chromium histidinate, over the course of treatment with the insulin-resistance inducing drug therapy.

### Example 6

### Treatment of Insulin Resistance with Chromium Histidinate

A subject is identified as having insulin resistance. The individual shows signs of decreased insulin function and/or hyperinsulinemia. The subject is administered between about 50 μg and 5000 μg chromium polyhistidinate daily, orally, in the form of a bar. After a period of time, the subject shows decreased hyperinsulinemia and improved insulin function.

### Example 7

### Treatment of Sexual Dysfunction with Chromium Complexes

A subject is identified as having impotence. The subject is orally administered between about 50 μg and 5000 μg chromium trihistidinate daily. After a period of time, the subject shows improved sexual function.

### Example 8

### Treatment of Cancer with Chromium Complexes

A subject is identified as having a solid tumor. The subject is administered between about 50 μg and 5000 μg chromium and histidine daily, parenterally. After a period of time, the metastasis of the subject's tumor tumor is reduced.

### Example 9

### Treatment of Cardiovascular Disease with Chromium Complexes

A subject is identified with cardiovascular disease. The subject shows signs of one or more conditions such as arteriosclerosis, atherosclerosis, peripheral vascular disease, or coronary heart disease. The subject is provided between about 50 μg and 5000 μg chromium histidinate daily. After a period of time, the subject's arteriosclerosis, atherosclerosis, peripheral vascular disease, or coronary heart disease improves.

### Example 9

### Treatment of Cardiovascular Disease with Combination Therapy

A subject is identified with cardiovascular disease. The subject shows signs of one or more conditions such as arteriosclerosis, atherosclerosis, peripheral vascular disease, or coronary heart disease. The subject is provided between about 50 μg and 5000 μg chromium histidinate daily. The subject is also provided a therapeutically effective amount of a second therapeutic for cardiovascular disease such as peripheral antiadrenergic therapy, antihypertensive drugs, vasodialtors, inodilators, cardiac glycosides, antidysrhythmic drugs. After a period of time, the subject's arteriosclerosis, atherosclerosis, peripheral vascular disease, or coronary heart disease improves.

### Example 11

### Treatment of Renal Disorders with Chromium Complexes

A subject is identified with compromised renal function. The subject shows one or more symptoms such as decreased creatinine clearance, elevated serum creatinine, decreased renal plasma flow, or decreased glomerular filtration rate. The subject is administered an effective amount of chromium trihistidinate daily, e.g. between 50 μg and 5000 μg chromium trihistidinate daily. After a period of time, the subject's renal function improves.

### Example 12

### Treatment of Glucose Metabolism Disorders with Chromium Complexes

A subject is identified with one or more glucose metabolism disorders such as diabetes or hyperglycemia. The subject is orally administered between about 50 μg and 5000 μg chromium polyhistidinate daily. After a period of time, the subject shows an improvement in fasting and/or post-prandial glucose levels.

### Example 13

### Treatment of Hypertension with Chromium Complexes

A subject is identified with hypertension, or having systolic blood pressure consistently 140 mmHg or greater, and/or diastolic blood pressure is consistently 90 mmHg or greater. The subject is administered between about 50 μg and 5000 μg chromium and histidine, orally, daily. After a period of time, the subject's hypertension is improved, e.g. the subject shows a decrease in blood pressure to normal levels.

### Example 14

### Treatment of PPAR disorders with Chromium Complexes

A subject is identified with a PPAR associated disorder. The subject has one or more of the following symptoms or conditions: rheumatoid arthritis, multiple sclerosis, inflammatory bowel disease, breast, colon, or prostate cancer, low levels of blood, lymph and/or cerebrospinal fluid apoE and/or apo A-1, elevated serum VLDL cholesterol levels, elevated serum LDL cholesterol levels, elevated triglyceride levels, elevated serum apo B levels, or the like. The subject is administered between about 50 μg and 5000 μg chromium histidinate, orally, daily. After a period of time, the subject's symptoms improve.

### Example 15

### Treatment of Dyslipidemia with Chromium Complexes

A subject is identified as having a dyslipidemia. The subject shows one or more symptoms such as elevated LDL cholesterol levels, decreased HDL levels, elevated total cholesterol levels, or elevated serum triglyceride levels. The subject is administered between about 50 μg and 5000 μg chromium histidinate daily, orally. After a period of time, the subject shows one or more of the following: decreased serum LDL cholesterol levels, increased serum HDL cholesterol levels, decreased total serum cholesterol levels, or decreased serum triglyceride levels.

The methods, compositions, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. Accordingly, it will be apparent to one skilled in the art that varying substitutions and modifications can be made to the invention disclosed herein without departing from the scope and spirit of the invention.

As used in the claims below and throughout this disclosure, by the phrase “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and can or can not be present depending upon whether or not they affect the activity or action of the listed elements.

Numerous literature and patent references have been cited in the present patent application. Each and every reference that is cited in this patent application is incorporated by reference herein in its entirety.

What is claimed is:

1. A method for reducing the risk of developing cardiometabolic syndrome in a subject in need thereof, comprising;
identifying a subject at risk of developing cardiometabolic syndrome; and
providing a therapeutically effective amount of a composition comprising chromium and histidine, a chromium histidinate complex, or a combination thereof to said subject.

2. The method of claim 1, wherein said identification step comprises identifying a subject with at least one condition selected from the group consisting of: insulin resistance, cardiovascular disease, progressive renal disease, end stage renal disease, endothelial dysfunction, left ventricular hypertrophy, cardiac hyperreactivity, dyslipidemia, hyperglycemia, enhanced rennin angiotensin activity, aldosterone syndrome, impaired pressure natriuresis, chronic low grade inflammation, diabetes mellitus, hypertension, atherosclerosis, micoralbuminuria, obesity, depression, Syndrome X, and polycystic ovary syndrome.

3. The method of claim 1, wherein said identification stem comprises identifying an individual that is taking a composition comprising a compound selected from the group consisting of: non-steroidal anti inflammatory compounds, oral contraceptives, implantable steroid contraceptives, hormone replacement therapy, beta blockers, potassium channel openers and immunosuppressive drugs.

4. The method of claim 1, wherein said composition further comprises a pharmaceutically acceptable carrier.

5. A method for reducing hyperglycemia and stabilizing the level of serum glucose comprising in a subject in need thereof, comprising:
identifying a subject in need of a reduction or stabilization in serum glucose levels; and
providing a therapeutically effective amount of a composition comprising chromium and histidine, a chromium histidinate complex, or a combination thereof to said subject.

6. The method of claim 5, wherein said composition further comprises a pharmaceutically acceptable carrier.

7. A method for treating dyslipidemia, comprising
identifying a subject with elevated triglyceride levels, elevated serum LDL levels, or reduced HDL levels; and
providing a therapeutically effective amount of a composition comprising chromium and histidine, a chromium histidinate complex, or a combination thereof to said subject.

8. The method of claim 7, wherein said composition further comprises a pharmaceutically acceptable carrier.

9. A method for lowering post prandial hyperglycemia in a subject in need thereof, comprising:
identifying a subject in need reduction in post-prandial glucose levels; and
providing a therapeutically effective amount of a composition comprising chromium and histidine, a chromium histidinate complex, or a combination thereof to said subject.

10. The method of claim 9, wherein said composition further comprises a pharmaceutically acceptable carrier.

11. A method for improving insulin sensitivity in a subject in need thereof, comprising:
identifying a subject in need of increased insulin sensitivity and providing a therapeutically effective amount of a composition comprising chromium and histidine, a chromium histidinate complex, or a combination thereof to said subject.

12. The method of claim 11, wherein said composition further comprises a pharmaceutically acceptable carrier.

13. A method for reducing free fatty acid levels in a subject in need thereof, comprising:
identifying a subject with elevated free fatty acid levels; and
providing a therapeutically effective amount of a composition comprising chromium and histidine, a chromium histidinate complex, or a combination thereof to said subject.

14. The method of claim 13, wherein said composition further comprises a pharmaceutically acceptable carrier.

15. A method for reducing cortisol levels in a subject in need thereof, comprising:
identifying a subject with elevated cortisol levels; and
providing a therapeutically effective amount of a composition comprising chromium and histidine, a chromium histidinate complex, or a combination thereof to said subject

16. The method of claim 15, wherein said composition further comprises a pharmaceutically acceptable carrier.
